(19) United States
(12) Patent Application Publication
(10) Pub. No.: US 2022/0025185 A1
(43) Pub. Date: Jan. 27, 2022

(54) SPECIFICALLY GLYCO-SUBSTITUTED PORPHYRINS AND CHLORINS FOR PHOTODYNAMIC THERAPY

(71) Applicants: UNIVERSITAT HAMBURG, Hamburg (DE); BIOLITEC UNTERNEHMENSBETEILIGUNGS II AG, Wien (AT)

(72) Inventors: Rene Klingenburg, Berlin (DE); Christian B. W. Stark, Hamburg (DE); Daniel Aicher, Berlin (DE); Arno Wiehe, Berlin (DE); Susanna Grafe, Jena (DE); Volker Albrecht, Nuthetal (DE)

(21) Appl. No.: 17/312,315

(22) PCT Filed: Dec. 10, 2019
(86) PCT No.: PCT/EP2019/084422

§ 371 Date: Jun. 9, 2021

(30) Foreign Application Priority Data
Dec. 10, 2018 (EP) 18211396.9
Dec. 10, 2018 (LU) LU101031

(51) Int. Cl. C09B 47/00; A61K 41/00; A61K 51/04

(52) U.S. Cl. CPC C09B 47/00; A61K 51/0485; A61K 41/0071

(57) ABSTRACT

The present invention provides certain tetrapyrrolic compounds having a structure of Formula (1), (2), or (3) wherein B is (I), (II) (III) or (IV), O—R¹ is a substituent in the meta or para position of the phenyl ring, R¹ is a glyco-substituent derived from a mono-, di-, or trisaccharide group, and each R² is independently selected from the group consisting of a linear or branched (fluoro-)alkyl group with 3 to 8 carbon atoms, phenyl, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30.

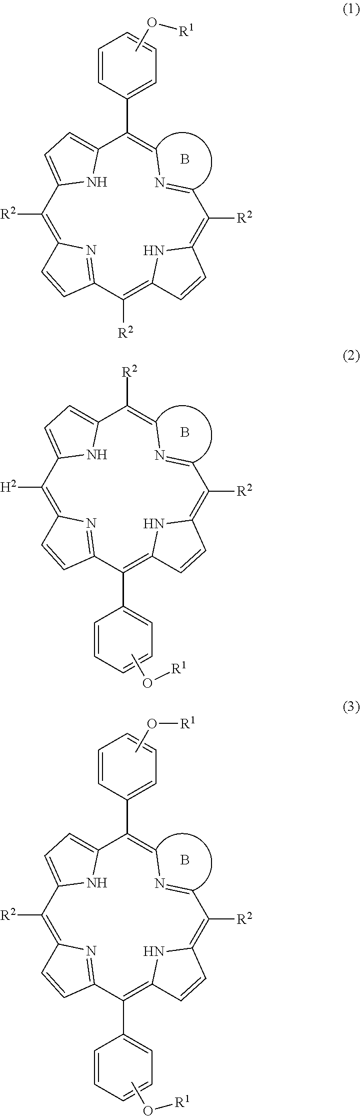

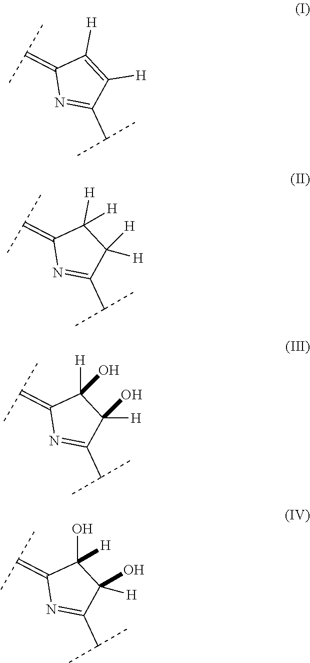

### FIELD OF THE INVENTION

The invention relates generally to photodynamic therapy, more particularly to specifically glyco-substituted porphyrins and chlorins to be used as photosensitizers for the treatment of hyperproliferative diseases, especially cancer.

### BACKGROUND OF THE INVENTION

Photodynamic therapy (PDT) is one of the most promising techniques being explored for use in a variety of medical applications (Photodynamic therapy, basic principles and clinical applications. Eds. B. W. Henderson, Th. J. Dougherty, Marcel Dekker, 1992, New York; A. P. Castano et al., Photodiagn. Photodyn. Ther. 2004, 1, 279-293; A. P. Castano et al., Photodiagn. Photodyn. Ther. 2005, 2, 1-23; R. R. Allison, C. H. Sibata, Photodiagn. Photodyn. Ther. 2010, 7, 61-75), and, particularly, is a well-recognized treatment for the destruction of tumors (Photodynamic tumor therapy. 2^{rd} and 3^{rd} generation photosensitizers. Ed. J. G. Moser, Harwood Academic Publishers, 1998, Amsterdam). Photodynamic therapy uses light and a photosensitizer (a dye) to achieve its desired medical effect. In principle, after light activation the triplet state of the photosensitizer is formed which interacts with neighboring molecules among them oxygen which is present in all cells. By this, reactive oxygen species, especially singlet oxygen, is formed. These reactive oxygen species damage cell components, leading eventually to cell death via apoptosis or necrosis. A large number of naturally occurring and synthetic dyes have been evaluated as potential photosensitizers for photodynamic therapy. Perhaps the most widely studied photosensitizers are the tetrapyrrolic macrocyclic compounds. Among them, especially porphyrins and chlorins have been tested for their PDT efficacy. Porphyrins are macrocyclic compounds with bridges of one carbon atom joining pyrroles to form a characteristic tetrapyrrole ring structure. There are many different classes of porphyrin derivatives including those containing dihydro-pyrrole units. Chlorins, as referred to in the present invention, are porphyrin derivatives containing one dihydro-pyrrole unit whereas bacteriochlorins are characterized by two dihydro-pyrrole units. In general, chlorins are characterized in that one double bond of the aromatic system in β-position is absent and bacteriochlorins are characterized in that two opposite double bonds are absent compared to the porphyrin. Methods to prepare chlorins are known in the art. They may e.g. be prepared by reduction of porphyrins (R. Bonnett et al., Biochem. J. 1989, 261, 277-280) or by oxidative dihydroxylation of porphyrins (C. Bruckner, D. Dolphin, Tetrahedron Lett. 1995, 36, 3295-3298; J. K. MacAlpine et al., J. Porphyrins Phthalocyanines 2002, 6, 146-155).

Examples of tetrapyrrolic macrocyclic compounds used as photosensitizers are described in US 2012/263,625 A1 from Aicher et al. which discloses glyco-substituted dihydroxy-chlorins for antibacterial PDT, U.S. Pat. No. 7,022,843 B1 from MacAlpine et al. which provides β,β′-dihydroxy meso-substituted chlorins as photosensitizers, and U.S. Pat. No. 7,166,719 B2 from Pandey et al. which discloses tetrapyrrole compounds containing a fluorinated substituent where the compound is a chlorin or a bacteriochlorin for PDT diagnostic and therapeutic application.

There are several properties that an effective photosensitizer should exhibit. Among them, a desirable characteristic in order to efficiently destroy deep target tissues is a strong absorption at long wavelength. Many current photosensitizers are not efficient enough as they have low absorption in the red region of the spectrum. Chlorins have the advantage that they possess an intense absorption in the red and near-infrared region of the electromagnetic spectrum. As light of longer wavelength penetrates deeper into the tissue, it is thus possible to treat e.g. more expanded tumors, if the PDT is employed for tumor therapy. Chlorins possessing potential for PDT can either be derived from natural sources or from total synthesis.

Another requirement for an effective photosensitizer is the accumulation in the tumor tissue. Moreover, this accumulation should occur within cell structures that are sensitive to the oxidative damage. In order to achieve this it is generally accepted that photosensitizers for tumor therapy have to be amphiphilic compounds which facilitates their accumulation in membrane structures of the cells.

In recent years the combination of tetrapyrrole photosensitizers with carbohydrates has intensively been investigated. One reason for this is that the decoration of the tetrapyrrole with carbohydrates is considered as a tool to increase their amphiphilicity (B. Chauvin et al., Eur. J. Pharm. Biopharm. 2013, 83, 244-252). Moreover, the connection of the photosensitizers with carbohydrates opens up the possibility to increase the photosensitizer accumulation in the tumor cells by specific interaction with receptors overexpressed in some types of malignant cells, such as lectin-type receptors (S. Ballut et al., Org. Biomol. Chem. 2012, 10, 4485-4495). For the connection of tetrapyrrole photosensitizers with carbohydrates a number of methods are known in the art, e.g. the glyco-substituted aldehydes may be condensed with pyrrole to form a glyco-substituted tetrapyrrole (P. Maillard et al., Tetrahedron Lett. 1992, 33, 8081-8084; K. Driaf et al., Tetrahedron Lett. 1993, 34, 1027-1030; D. Oulmi et al., J. Org. Chem. 1995, 60, 1554-1564; Y. Mikata et al., Tetrahedron Lett. 1998, 39, 4505-4508; I. Laville et al., Bioorg. Med. Chem. 2004, 12, 3673-3682) or the glycosylation may be performed on the final tetrapyrrole (see e.g.: G. Fulling et al., Angew. Chem. nt. Ed. Engl. 1989, 28, 1519-1521; J. P. C. Tome et al., Bioorg. Med. Chem. 2005, 13, 3878-3888; D. Aicher et al., Synlett 2010, 395-398). These methods for preparing glycosylated porphyrins may be combined with the above-mentioned methods of preparing chlorins to obtain glycosylated chlorins or bacteriochlorins (I. Laville et al., Bioorg. Med. Chem. 2003, 11, 1643-1652).

In the embodiments described in the art it is emphasized that tri- and tetraglycosylated or even higher glycosylated tetrapyrroles are especially active as photosensitizers (I. Laville et al., Bioorg. Med. Chem. 2003, 11, 1643-1652; Y. Mikata et al., Tetrahedron Lett. 1998, 39, 4505-4508; Laville, S. et al. Bioorg. Med. Chem. 2004, 12, 3673-3682; I. Laville et al., Bioorg. Med. Chem. 2003, 11, 1643-1652; S. Ballut et al., Org. Biomol. Chem. 2012, 10, 4485-4495; B. Chauvin et al., Eur. J. Pharm. Biopharm. 2013, 83, 244-252).

### Objectives and Brief Summary of the Invention

Nevertheless, there is still a desire for biologically active compounds that can be used as highly effective photosensitizers for a wide range of applications including light irradiation treatments, such as photodynamic therapy of cancer and other diseases, as well as for pharmaceutical compositions of such biologically active compounds. In particular, upon light irradiation the compounds should exhibit a strong phototoxicity rendering them suitable as photosensitizer for PDT. Furthermore, the compounds should easily be synthesized.

These objects are surprisingly achieved by a tetrapyrrolic compound according to claims 1 to 12, a composition according to claims 13 to 15 and the use of the tetrapyrrolic compound according to claims 16 and 17.

The tetrapyrrolic compounds according to the invention have a structure of Formula 1, 2 or 3:

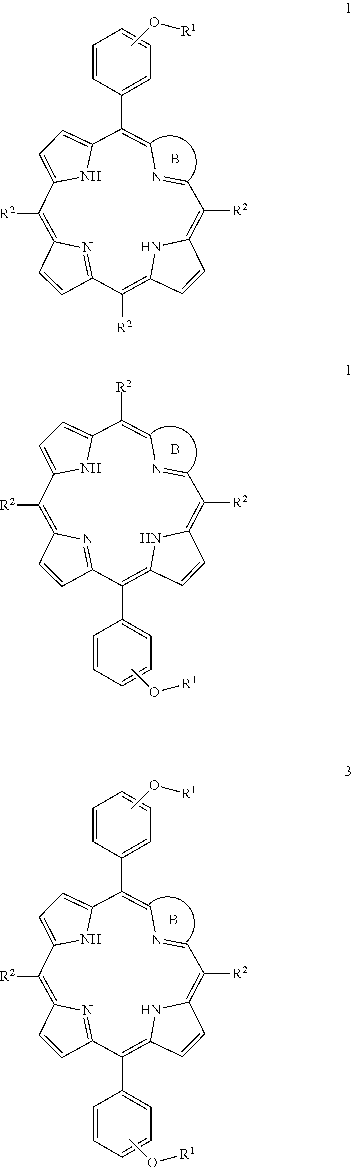

wherein B is

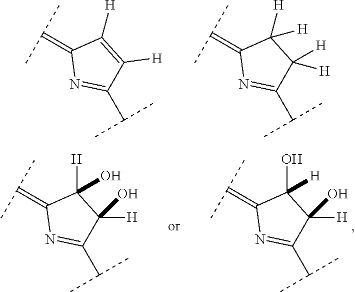

O-R¹ is a substituent in the meta or para position of the phenyl ring,

R¹ is a glyco-substituent derived from a mono-, di-, or trisaccharide group, and

each R² is independently selected from the group consisting of a linear or branched (fluoro-)alkyl group with 3 to 8 carbon atoms, phenyl, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30,

wherein
(a) in Formula 1 or 2, if B is

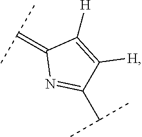

O—R¹ is a substituent in the para position of the phenyl ring and R¹ is glucosyl, then each R² is independently selected from the group consisting of a linear or branched alkyl group with 5 to 8 carbon atoms, a linear or branched fluoroalkyl group with 3 to 8 carbon atoms, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)˜CH₃ with n=1-30,
(b) in Formula 1 or 2, if B is

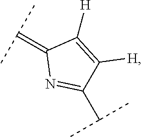

O—R¹ is a substituent in the para position of the phenyl ring and R¹ is galactosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30,
(c) in Formula 1 or 2, if O-R¹ is a substituent in the meta position of the phenyl ring and R¹ is glucosyl and R² is n-hexyl, phenyl or 3,5-bis(trifluoromethyl)phenyl, then B is

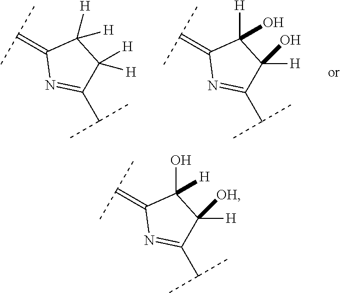

(d) in Formula 1 or 2, if O-R¹ is a substituent in the meta position of the phenyl ring and R¹ is galactosyl and R² is phenyl, then B is

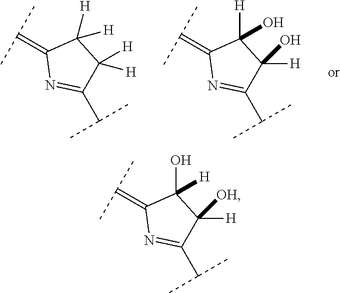

### (e) in Formula 3, if B is

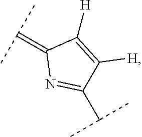 [0023] O—R¹ is a substituent in the para position of the phenyl ring and R¹ is glucosyl, then each R² is independently selected from the group consisting of a linear or branched (fluoro-)alkyl group with 5 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30,
(f) in Formula 3, if B is

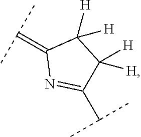

O—R¹ is a substituent in the meta position of the phenyl ring and R¹ is glucosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30, and
(g) in Formula 3, if B is

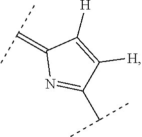

O—R¹ is a substituent in the para position of the phenyl ring and R¹ is galactosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30.

Formulae 1, 2 and 3 as well as other formulae shown herein cover all stereoisomeric forms as well as mixtures of different stereoisomeric forms, such as e.g. racemates. The formulae cover only those compounds that are compatible with the chemical valence theory.

### DETAILED DESCRIPTION

The biologically active tetrapyrrolic compounds of the present invention can be used as photosensitizer for a wide range of light irradiation treatments such as photodynamic therapy (PDT) of cancer and other hyperproliferative diseases.

Tetrapyrrolic compounds according to Formula 1, 2 or 3 are preferred, wherein
(a) in Formula 1 or 2, if B is

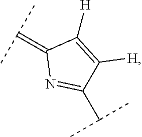

O—R¹ is a substituent in the para position of the phenyl ring and R¹ is glucosyl, then each R² is independently selected from the group consisting of a linear or branched alkyl group with 5 to 8 carbon atoms, a linear or branched fluoroalkyl group with 3 to 8 carbon atoms, 3,5-bis(trifluoromethyl)phenyl, 4-(1′-thio-β-D-glucosyl)-2,3,5,6-tetrafluorophenyl, and 4-(1′-thio-β-D-galactosyl)-2,3,5,6-tetra-fluorophenyl,
(b) in Formula 1 or 2, if B is

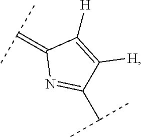

O—R¹ is a substituent in the para position of the phenyl ring and R¹ is galactosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, and 4-(1′-thio-β-D-galactosyl)-2,3,5,6-tetrafluorophenyl; more preferably each R² is independently selected from the group consisting of a linear or branched (fluoro-)alkyl group with 5 to 8 carbon atoms, 3,5-bis(trifluoromethyl)phenyl, 4-(1′-thio-β-D-glucosyl)-2,3,5,6-tetrafluorophenyl, and 4-(1′-thio-β-D-galactosyl)-2,3,5,6-tetrafluorophenyl,
(e) in Formula 3, if B is

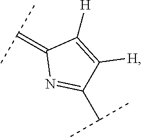

O—R¹ is a substituent in the para position of the phenyl ring and R¹ is glucosyl, then each R² is independently selected from the group consisting of a linear or branched (fluoro-)alkyl group with 5 to 8 carbon atoms, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl,
(f) in Formula 3, if B is

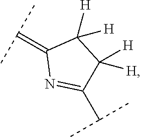

O—R¹ is a substituent in the meta position of the phenyl ring and R¹ is glucosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, and 4-(1′-thio-β-D-galactosyl)-2,3,5,6-tetrafluorophenyl, or
(g) in Formula 3, if B is

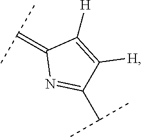

O—R¹ is a substituent in the para position of the phenyl ring and R¹ is galactosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl.

Furthermore, it is preferred that tetrapyrrolic compounds of Formula 3 do not comprise a linear or branched (fluoro-)alkyl group with 3 to 8 carbon atoms. In particular, if in Formula 3 B is

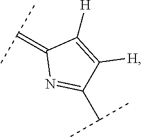

O—R¹ is a substituent in the meta position of the phenyl ring and R¹ is glucosyl, then R² is not linear or branched (fluoro-)alkyl group with 3 to 8 carbon atoms, like n-hexyl.

Accordingly, it is preferred that in Formula 3 R² is independently selected from phenyl, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30.

It is particularly preferred that the tetrapyrrolic compounds according to the invention have a structure of Formula 1 or 2 as described herein.

Preferred compounds of Formula 1, 2 or 3 are:

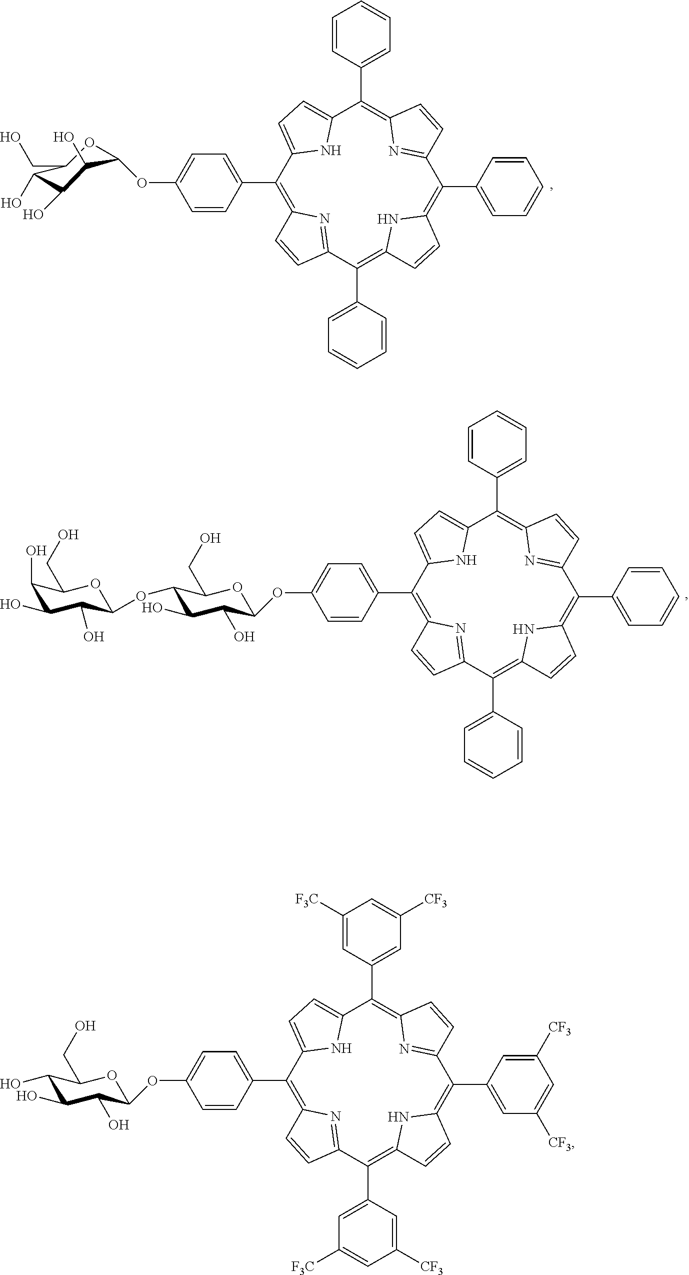

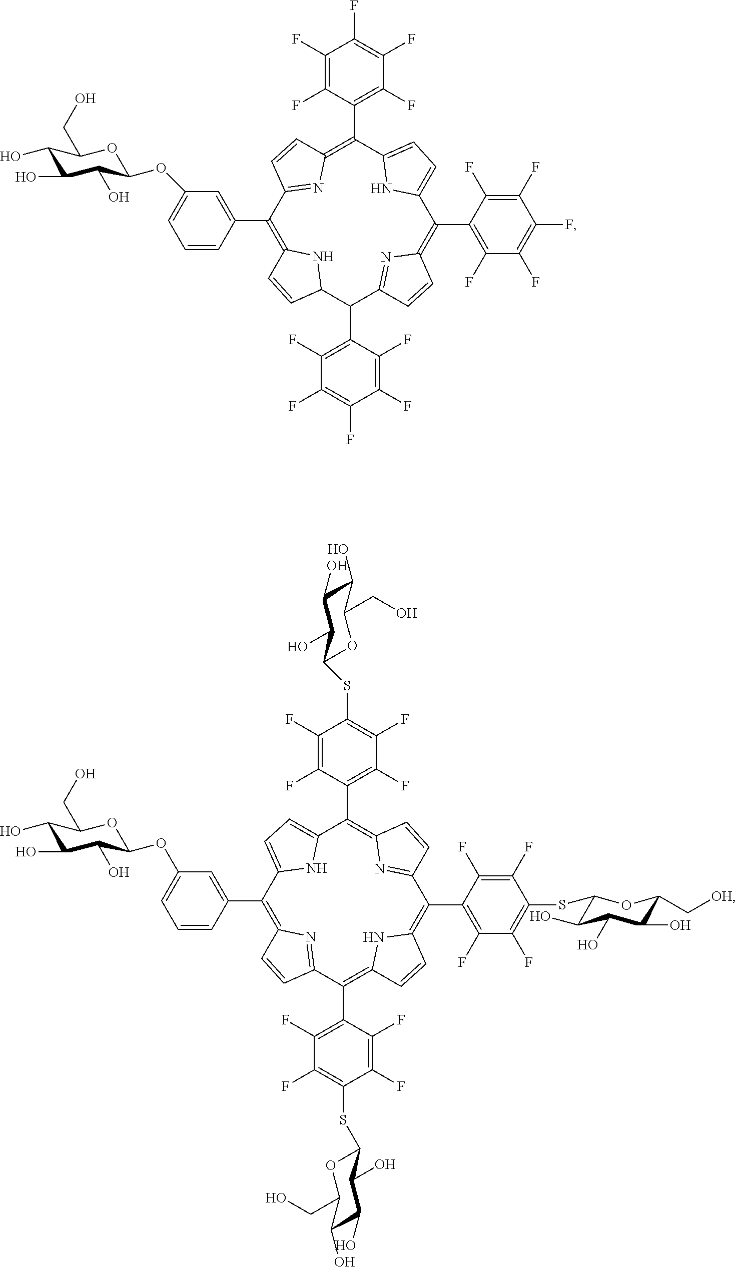

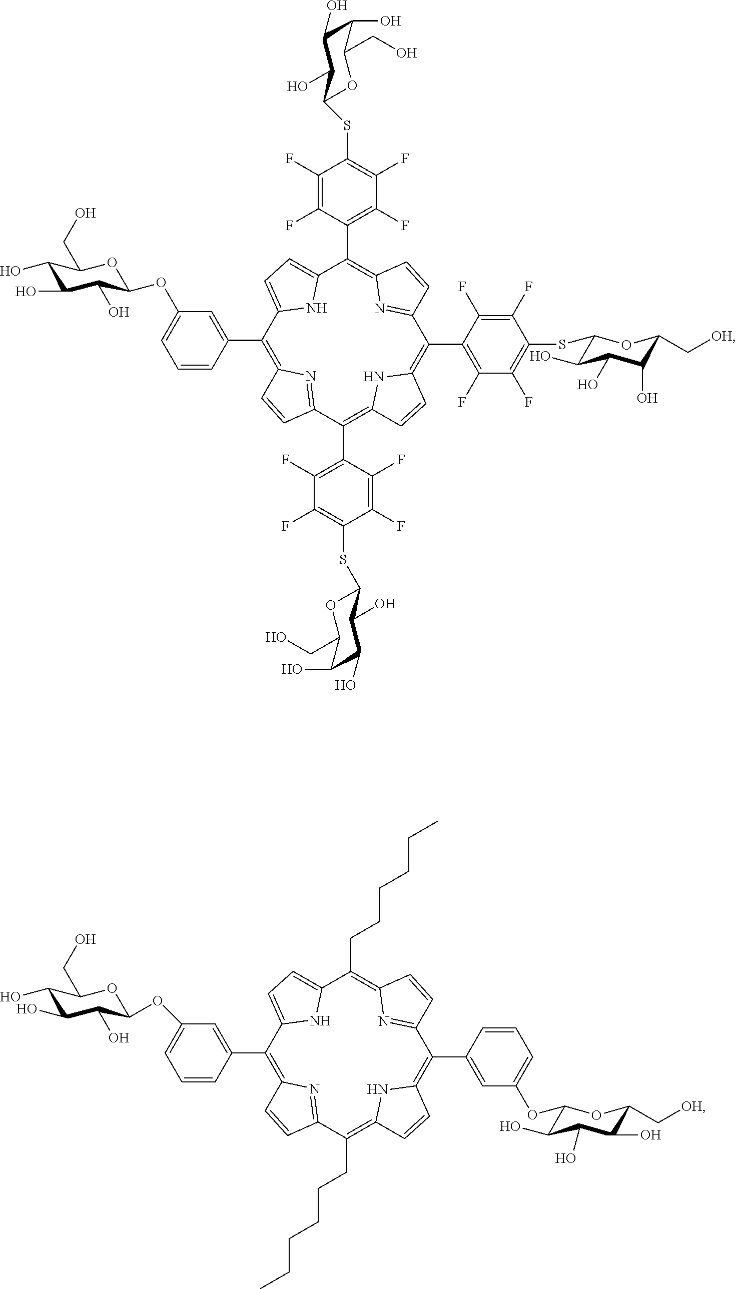

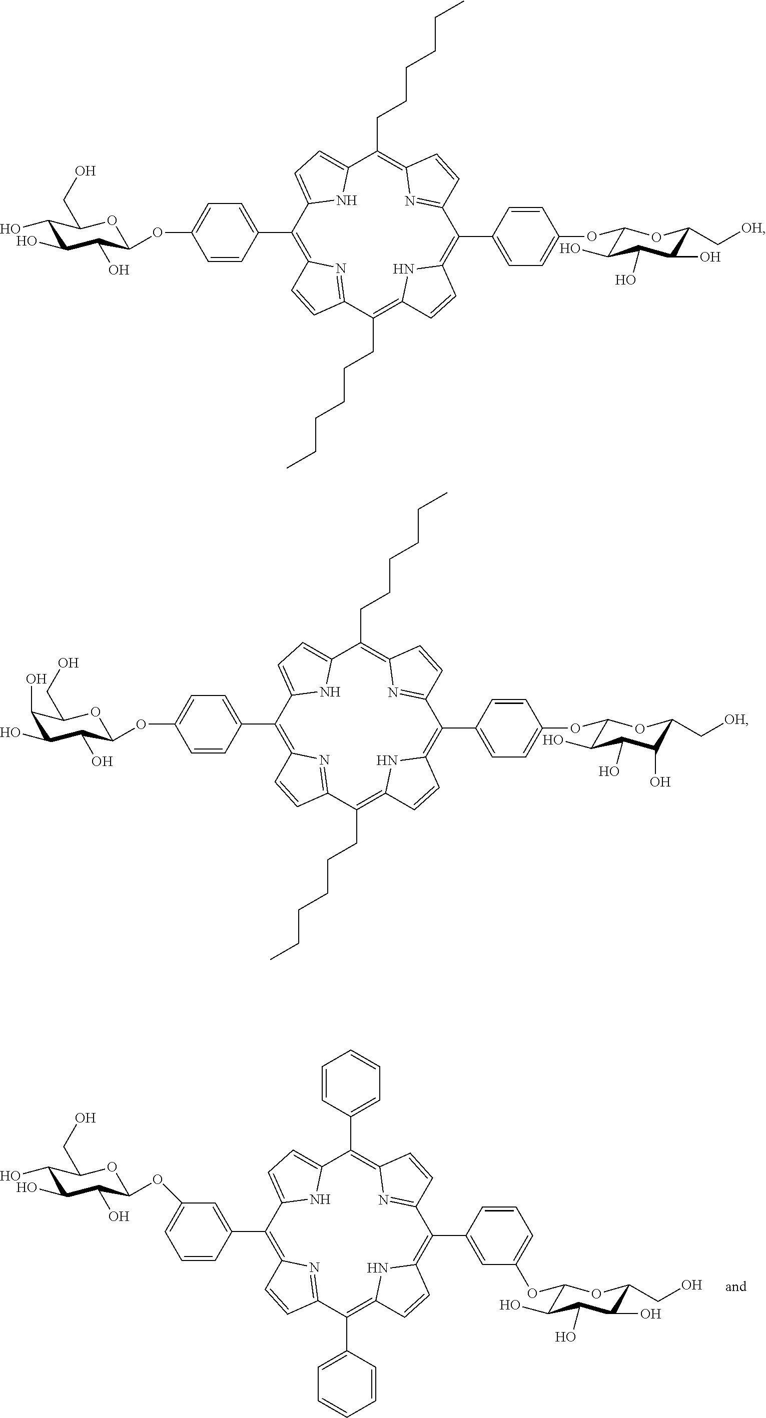

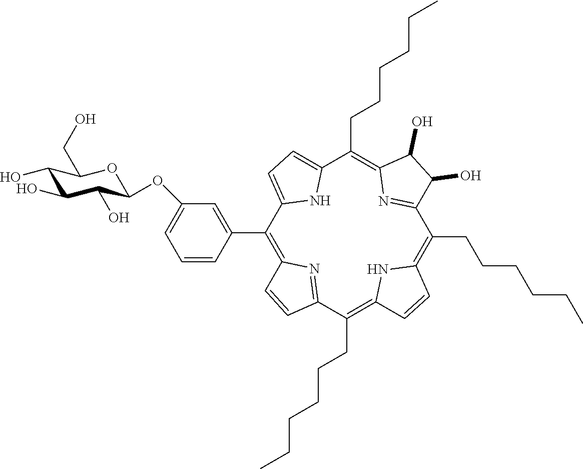

Particularly preferred compounds of Formula 1, 2 or 3 are:

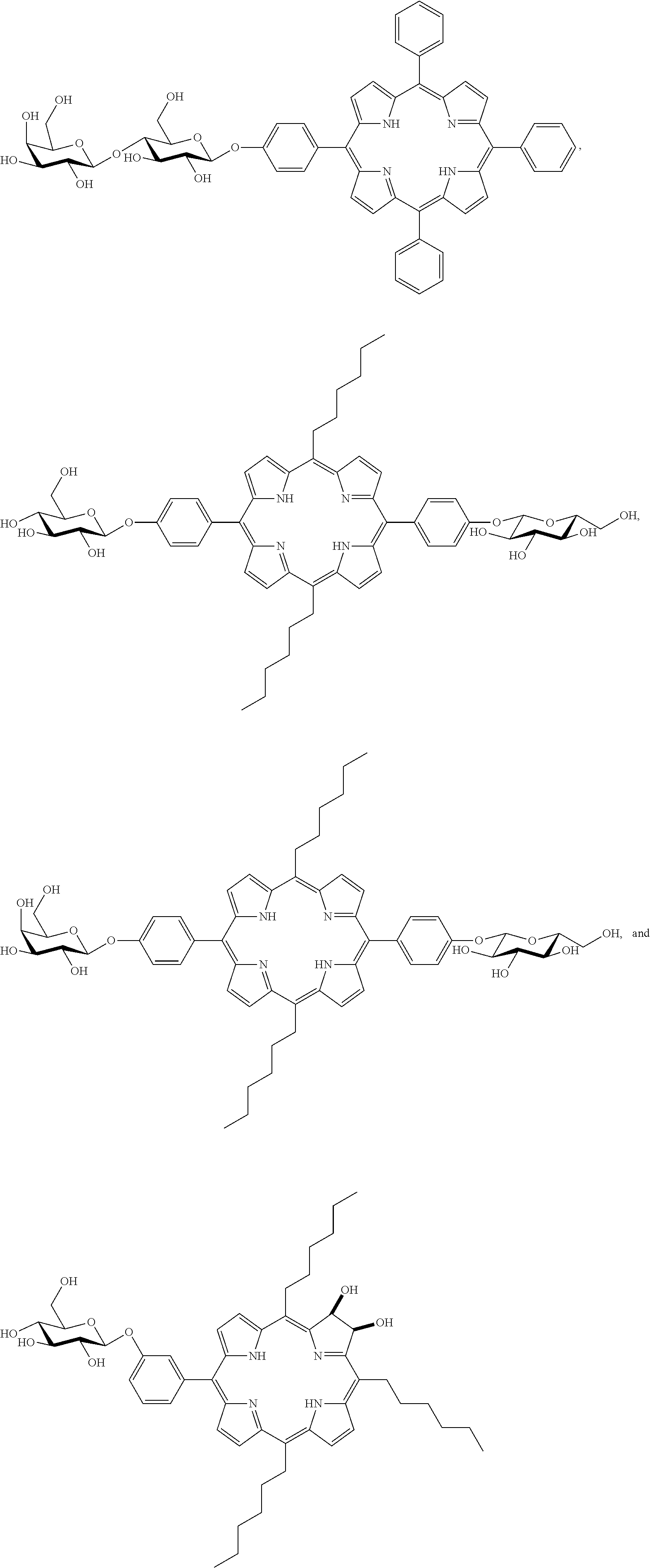

The compounds according to the invention are tetrakis-meso-substituted porphyrin and chlorin structures and it has unexpectedly been found that various porphyrins and chlorins containing one or two specific glycosylated residues in their meso positions are especially suited for such a medical application. They exhibit an unusually strong PDT activity compared to the corresponding tri- and tetraglycosylated tetrapyrroles, although the latter are usually considered as better photosensitizers. Furthermore, the new photosensitizers provided by the present invention have the advantage that they can easily be produced and characterized. Moreover, as the present invention provides methods to tailored amphiphilic compounds for desired PDT applications, target tissue selectivity and, therefore, PDT efficacy, is increased.

R¹ is a glyco-substituent derived from a mono-, di-, or trisaccharide group. In particular, R¹ is a glyco-substituent selected from glycosyl groups of mono-, di-, or trisaccharides. In a preferred embodiment the glyco-substituent comprises a glycosyl group of a mono- or disaccharide derived from or consisting of naturally occurring monosaccharides or disaccharides as building blocks, such as in particular glucose, galactose, mannose, ribose, fructose, rhamnose, lactose, partially deoxygenated derivatives thereof, aminosugars, such as glucosamines or galactosamines, neuraminic acids and combinations thereof. In a particularly preferred embodiment, R¹ is a glycosyl group of a mono- or disaccharide, wherein the mono- or disaccharide is preferably selected from the group consisting of glucose, galactose, mannose, ribose, fructose, rhamnose, lactose, partially deoxygenated derivatives thereof, aminosugars, such as glucosamines or galactosamines, neuraminic acids and combinations thereof.

Furthermore, in a preferred embodiment each R² is independently selected from the group consisting of a linear or branched (fluoro-)alkyl group having 3 to 8 carbon atoms, phenyl, pentafluorophenyl and 3,5-bis(trifluoromethyl)phenyl. Moreover, in a preferred embodiment each R² group of a compound according to the invention is the same R² group.

In a further preferred embodiment R¹ is mannosyl or lactosyl and R² is a linear or branched (fluoro-)alkyl group with 3 to 8 carbon atoms, phenyl, pentafluorophenyl, 3,5-bis(trifluoromethyl)phenyl, 4-(1′-thio-β-D-glucosyl)-2,3,5,6-tetrafluorophenyl or 4-(1′-thio-β-D-galactosyl)-2,3,5,6-tetrafluorophenyl.

In another preferred embodiment B is

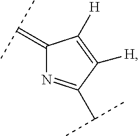

O-R¹ is a substituent in the para position of the phenyl ring, R¹ is mannosyl or lactosyl and R² is phenyl.

In yet another preferred embodiment in Formula 1 or 2 O-R¹ is a substituent in the meta position of the phenyl ring, R¹ is glucosyl and each R² is a linear or branched fluoroalkyl group with 3 to 8 carbon atoms, 3,5-bis(trifluoromethyl)phenyl, 4-(1′-thio-β-D-glucosyl)-2,3,5,6-tetrafluorophenyl or 4-(1′-thio-β-D-galactosyl)-2,3,5,6-tetrafluorophenyl.

In a further preferred embodiment in Formula 3 B is

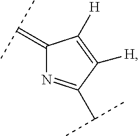

R¹ is glucosyl or galactosyl and each R² is a linear or branched (fluoro-)alkyl group with 5 to 8 carbon atoms, pentafluorophenyl, 3,5-bis(trifluoromethyl)phenyl, 4-(1′-thio-β-D-glucosyl)-2,3,5,6-tetrafluorophenyl or 4-(1′-thio-β-D-galactosyl)-2,3,5,6-tetrafluorophenyl.

In another preferred embodiment in Formula 1 or 2 B is

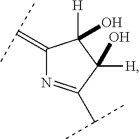

R¹ is glucosyl or galactosyl and each R² is a linear or branched (fluoro-)alkyl group with 5 to 8 carbon atoms, pentafluorophenyl, 3,5-bis(trifluoromethyl)phenyl, 4-(1′-thio-β-D-glucosyl)-2,3,5,6-tetrafluorophenyl or 4-(1′-thio-β-D-galactosyl)-2,3,5,6-tetrafluorophenyl.

In a particular preferred embodiment in Formula 1 or 2 B is

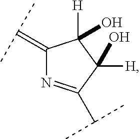

R¹ is glucosyl and each R² is a linear or branched alkyl group with 5 to 8 carbon atoms.

In another preferred embodiment of the present invention O-R¹ is a glycosidic bond. In particular, a glycosidic bond is formed between the hemiacetal or hemiketal group of a saccharide or a molecule derived from a saccharide and the hydroxyl group of a precursor of the tetrapyrrolic compound, such as an alcohol.

In a further preferred embodiment of the present invention R² is 4-(1′-thio-β-D-glucosyl)-2,3,5,6-tetrafluorophenyl or 4-(1′-thio-β-D-galactosyl)-2,3,5,6-tetrafluorophenyl.

In a particularly preferred embodiment of the present invention in Formula 1 or 2 R¹ is glucosyl and R² is 4-(1′-thio-β-D-glucosyl)-2,3,5,6-tetrafluorophenyl or 4-(1′-thio-β-D-galactosyl)-2,3,5,6-tetrafluorophenyl.

The tetrapyrrolic compounds according to Formula 1, 2 or 3 have two or three R² substituents which can be the same or different. In a preferred embodiment, each R² of the compound according to the invention is the same.

Moreover, the tetrapyrrolic compounds according to Formula 3, which have a ‘trans’ arrangement of meso-glyco-substituents, have two R¹ substituents which can be the same or different. In a preferred embodiment, each R¹ of a compound of Formula 3 is the same.

The tetrapyrrolic compounds according to the invention can be prepared by methods generally known in the art.

For example, tetrapyrrolic compounds according to the invention can easily be synthesized by reacting a hydroxyphenyl-substituted tetrapyrrole with a corresponding glyco-trichloroacetimidate.

In one embodiment of the present invention tetrapyrrolic compounds are provided combining two different kinds of glyco-substituents R¹. For this purpose the glycosylation of tetrapyrroles via trichloroacetimidates as described in Aicher et al. (D. Aicher et al., Synlett 2010, 395-398) is combined with a nucleophilic aromatic substitution on pentafluorophenyl-substituted tetrapyrroles known from e.g. X. Chen et al., Biochem. 2004, 43, 10918-10929; S. Hirohara et al., Bioorg. Med. Chem. 2010, 18, 1526-1535; C. R. Becer et al., Macromolecules 2009, 42, 2387-2394. 1-thio-β-D-glucose, 1-thio-β-D-galactose, 1-thio-α-D-mannose and their derivates bearing protective groups, such as acetyl groups, are preferred nucleophiles for the nucleophilic aromatic substitution on pentafluorophenyl residues. 1-thio-β-D-glucose, 1-thio-β-D-galactose and their derivates bearing protective groups, such as acetyl groups, are particularly preferred.

In general, the novel photosensitizers having a structure of Formula 1, 2 or 3 according to the present invention can be synthesized by functionalizing tetrapyrrole compounds with the desired glyco-substituents (D. Aicher et al., Synlett 2010, 395-398). These glyco-modified compounds can further be converted to simple chlorins or dihydroxy-chlorins (see EP 0337601 B1; WO 09/613504 A1, WO 00/061584 A1; C. Bruckner, D. Dolphin, Tetrahedron Lett. 1995, 36, 3295-3298; C. Bruckner, D. Dolphin, Tetrahedron Lett. 1995, 36, 9425-9428; H. W. Daniell et al., Tetrahedron Lett. 2003, 44, 4045-4049; F. Rancan et al., J. Photochem. Photobiol. B: Biology 2005, 78, 17-28; D. Aicher et al., Bioorg. Med. Chem. Lett. 2011, 21, 5808-5811).

As used herein, mono- and di-glycosylated tetrapyrrolic compounds according to the invention are also referred to as unsymmetrical porphyrins and chlorins. Acceptable starting materials for the synthesis of the unsymmetrical porphyrins and chlorins according to the present invention can be pyrrole and aldehydes. More specifically, pyrrole and two aldehydes are typically employed for the synthesis of the unsymmetrically substituted porphyrins. In particular, pyrrole and aldehydes are subjected to a condensation reaction. Suitable methods for this condensation are known in the art (J. S. Lindsey et al., J. Org. Chem. 1987, 52, 827-836). Alternatively, the unsymmetrically substituted porphyrins can also be synthesized using di- or tripyrromethanes and aldehydes, as is also known in the art (C.-H. Lee et al., Tetrahedron 1994, 50, 11427-11440). After condensation, purification and deprotection at their hydroxyl groups the desired unsymmetrically substituted porphyrins are modified at their hydroxyphenyl substituents (either 3-hydroxyphenyl or 4-hydroxyphenyl) with the glyco-trichloroacetimidates as glycosyl donors. After purification of the modified porphyrins, these can, if desired, be converted to the corresponding chlorins.

In one embodiment of the present invention a glyco-substituted porphyrin is synthesized and converted to the corresponding chlorin system by dihydroxylation or reduction, preferably dihydroxylation. Dihydroxylation using osmium tetroxide is particularly preferred.

In a specifically preferred embodiment of the present invention a porphyrin of the ‘trans’-A₂B2-type is synthesized, having a glyco-substituent as substituent A and an alkyl or fluoroalkyl groups or (substituted) phenyl rings as substituent B. This porphyrin again can easily be converted to the chlorin and the dihydroxychlorin with the methods known in the art.

Furthermore, the present invention is directed to a pharmaceutical composition comprising a tetrapyrrolic compound according to the invention.

The tetrapyrrolic compounds disclosed in the present invention are mostly lipophilic compounds because such compounds have a higher tendency to accumulate in cellular membrane structures. It is in these membrane structures where the reactive oxygen species generated by the photodynamic treatment can effectively damage the (tumor) cells. However, due to their lipophilic nature photosensitizers are sparingly or not at all water soluble so suitable pharmaceutical formulations are needed for their clinical application. Such pharmaceutical formulations may involve liposomal, nanoparticle or polymer-based formulations. For increasing patient compliance during treatment improved formulations of photosensitizers e.g. oral formulations are needed. Accordingly, liposomal formulations comprising the tetrapyrrolic compound according to the invention are preferred. Liposomal formulations comprising the tetrapyrrolic compound according to the invention and further comprising PLGA particles, HSA particles, cyclodextrines and/or polymer particles are particularly preferred. Liposomal formulations as stated above conjugated to a targeting agent, preferably an antibody or a fragment thereof, are most preferred.

In this respect, WO 2011/071970 A2 by Langer et al. discloses suitable photosensitizer formulations based on poly-lactic-co-glycolic-acid (PLGA) whereas WO 2011/071968 A2 by Langer et al. discloses formulations based on human serum albumin (HSA) nanoparticles. Furthermore, WO 2005/023220 A1 by Albrecht et al. discloses suitable liposomal formulations. Possible oral formulations for such photosensitizers are described in WO 2010/129337 A2 by Graefe et al. and in WO 2010/129340 A2 by Farmer et al.

Typically, PDT is accomplished by first incorporating the compound according to the invention into a pharmaceutically acceptable application vehicle (e.g. ethanolic solution, liposomal formulation or a formulation based on HSA or PLGA particles) for delivery of the tetrapyrrolic compound to a specific treatment site. After administering the tetrapyrrolic compound in the vehicle to a treatment area, sufficient time is allowed so that the tetrapyrrolic compound preferentially accumulates in the diseased tissue. Lastly, the treatment area is irradiated with light of a proper wavelength and sufficient power to activate the porphyrin derivatives to induce necrosis or apoptosis in the cells of said diseased tissue. Thus, one of the main advantages is that convenient pharmaceutical formulations can be created for the biologically active tetrapyrrolic compounds of the present invention such as liposomal formulation to be injected avoiding undesirable effects like precipitation at the injection site or delayed pharmacokinetics of the tetrapyrrole systems. Due to their amphiphilic nature, the chemically stable porphyrin and chlorin derivatives of the present invention can be prepared in various pharmaceutically acceptable and active preparations for different administration methods, e.g. injections. In a specifically preferred embodiment such amphiphilic compounds are formulated into liposomes. This liposomal formulation can then be injected avoiding undesirable effects such as precipitation at the injection site or delayed pharmacokinetics of the tetrapyrrole systems.

Hence, the tetrapyrrolic compounds according to the invention are suitable for use in medical applications such as photodynamic therapy, in particular photodynamic therapy of tumors and other hyperproliferative diseases, dermatological disorders, ophthalmological disorders, urological disorders, arthritis and other inflammatory or hyperproliferative diseases.

Accordingly, a method of photodynamic therapy, in particular photodynamic therapy of tumors and other hyperproliferative diseases, dermatological disorders, ophthalmological disorders, urological disorders, arthritis and other inflammatory or hyperproliferative diseases comprising administering a tetrapyrrolic compound according to the invention or a pharmaceutical composition thereof to a patient in need thereof, is also disclosed herein.

Furthermore, the tetrapyrrolic compounds according to the invention are suitable for use in diagnosis, in particular fluorescence diagnosis.

The following examples are presented to provide those of ordinary skill in the art with a full and illustrative disclosure and description of how to make the porphyrin and chlorin derivatives of the invention and show their photodynamic activity and are not intended to limit the scope of what the inventor regards as the invention. Efforts have been made to ensure accuracy with respect to numbers used (e.g. amounts, temperature etc.), but some experimental errors and deviations should be accounted for. Also, best measures have been taken to name the compounds with their systematic IUPAC name, nevertheless the basic reference are the given structural formulas based on the experimental spectroscopic data.

### BRIEF DESCRIPTION OF FIGURES

FIG. 1 shows the results of the cell test of 5,15-bis-(4-β-D-glucosylphenyl)-10,20-dihexylporphyrin with the cell lines A431, CAL-27, L929 and HT29 (Example 4.1).

FIG. 2 shows the results of the cell test of 5,15-bis-(4-β-D-galactosylphenyl)-10,20-dihexylporphyrin with the cell lines A431, A253, CAL-27, L929 and HT29 (Example 4.2).

FIG. 3 shows the results of the cell test of 5-(4-β-D-lactosylphenyl)-10,15,20-triphenylporphyrin with cell lines A431, A253, CAL-27, L929 and HT29 (Example 4.3).

FIG. 4 shows the results of the cell test of 5-(3-β-D-glucosylphenyl)-10,15,20-trihexyl-17,18-dihydroxy-17,18-chlorin with the cell lines A431 and CAL-27 (Example 4.4).

FIG. 5 shows the results of the cell test of 5-(3-β-D-glucosylphenyl)-10,15,20-tris-(4-1′-thio-β-D-glucosyl-2,3,5,6-tetrafluorophenyl)-porphyrin with the cell lines A 253, CAL-27, L929 (Example 4.5).

FIG. 6 shows the results of the cell test of 5,10,15-tris-(3-β-D-galactosylphenyl)-20-[3,5-bis-(trifluoromethyl)-phenyl]-17,18-dihydroxy-17,18-chlorin with the cell line HT29 (Reference Example 4.6).

FIG. 7 shows the results of the cell test of 5,10,15-tris-(3-β-D-lactosylphenyl)-20-[3,5-bis-(trifluoromethyl)-phenyl]-17,18-dihydroxy-17,18-chlorin with the cell line HT29 (Reference Example 4.7).

FIG. 8 shows the results of the cell test of 5,10,15,20-tetrakis-(3-β-D-galactosyl)-porphyrin with the cell lines A431, A253, CAL-27, L929 and HT29 (Reference Example 4.8).

FIG. 9 shows the results of the cell test of 5,10,15,20-tetrakis-(4-β-D-glucosyl)-porphyrin with the cell lines A431, A253 and HT29 (Reference Example 4.9).

### EXAMPLES

All reagents were used as purchased from commercial suppliers. Dichloromethane was purified by distillation over K₂CO₃ prior to use. Thin layer chromatography (TLC) was performed using Merck silica gel 60 (without fluorescence indicator) pre-coated on aluminum sheets. Flash chromatography was carried out using Fluka silica gel 60, 0.040-0.063 mm (230-400 mesh). ¹H and ¹³C NMR spectra were recorded in CDCl₃, (CD₃)₂CO, CD₃OD or (CD₃)₂SO on Bruker (AC 500 and AVIII 700) and JOEL (Eclipse 500) instruments. Chemical shifts δ are given in ppm relative to TMS as internal standard or relative to the resonance of the residual solvent peak, J values are given in Hz. Mass spectra were recorded on an Agilent 6210 ESI-TOF, Agilent Technologies, Santa Clara, Calif., USA. Electronic absorption spectra were recorded on a Specord S300 (Analytik Jena) spectrophotometer using dichloromethane, ethanol, acetone or dimethyl sulfoxide as solvent.

### Example 1—Preparation of Glycosubstituted Porphyrins

### 1.1 Preparation of 5-[4-(2,3,4,6-tetraacetyl-α-D-mannosyl)phenyl]-10,15,20-triphenylporphyrin

In a typical experiment, under argon atmosphere, Zn(II)-5-(4-hydroxyphenyl)-10,15,20-triphenylporphyrin (100 mg, 144 μmol) was dissolved in 20 ml dry dichloromethane and 0.5 ml dry acetonitrile. Then, 2,3,4,6-tetraacetyl-α-D-mannose trichloroacetimidate (862 mg, 1.75 mmol) and BF₃-Et₂O (7.5 μl, 60 μmol) were added. After stirring for 3 hours, the mixture was transferred to a separatory funnel. The organic layer was washed with water (2×100 ml) and the solvent was evaporated under reduced pressure. To remove the zinc, the residue was dissolved in 20 ml tetrahydrofuran, and 0.6 ml of hydrochloric acid (25%) were added. After stirring for 10 minutes, water (100 ml) and dichloromethane (150 ml) were added. The organic layer was separated and washed with water (2×100 ml). After drying with Na₂SO₄, the solvent was evaporated under reduced pressure. Further purification was achieved by flash chromatography, using dichloromethane/methanol 95:5 as the eluent. The analytically pure product (108 mg, 78%) was obtained as a violet crystalline solid after recrystallization from dichloromethane/aqueous methanol.

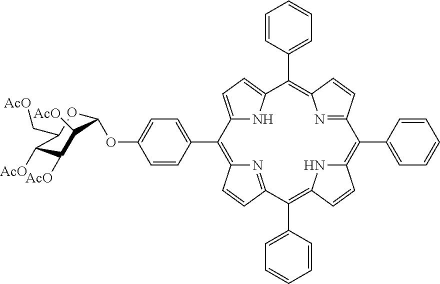

mp: >300° C., ¹H NMR (700 MHz, (CD₃)₂CO): δ=−2.73 (br s, 2H, NH), 2.04 (s, 3H, OAc), 2.06 (s, 3H, OAc), 2.13 (s, 3H, OAc), 2.24 (s, 3H, OAc), 4.25 (dd, J=2.8, 12.3 Hz, 1H, H-6_{A}‘ose’), 4.37 (dd, J=6.4, 12.3 Hz, 1H, H-6_{B}‘ose’), 4.45 (ddd, J=2.8, 6.4, 10.0 Hz, 1H, H-5‘ose’), 5.46 (dd, J=10.0, 10.0 Hz, 1H, H-4‘ose’), 5.64 (dd, J=1.9, 3.8 Hz, 1H, H-2‘ose’), 5.69 (dd, J=3.8, 10.0 Hz, 1H, H-3‘ose’), 6.02 (d, J=1.9 Hz, 1H, H-1 ‘ose’), 7.62 (d, J=8.7 Hz, 2H, Ar—Hₘₑₜₐ), 7.79-7.82 (m, 9H, 6×Ph-Hₘₑₜₐ, 3×Ph-Hₚₐᵣₐ), 8.21 (d, J=8.7 Hz, 2H, Ar—Hₒᵣₜₕₒ), 8.23-8.25 (m, 6H, Ph-Hₒᵣₜₕₒ), 8.86 (s, 6H, β-H), 8.90 (d, J=4.3 Hz, 2H, β-H) ppm. ¹³C NMR (176 MHz, (CD₃)₂CO): 6=21.69 (q, OCH₃), 21.73 (q, OCH₃), 21.76 (q, OCH₃), 21.80 (q, OCH₃), 64.21 (t, C-6‘ose’), 67.86 (d, C-4‘ose’), 70.96 (d, C-3‘ose’), 71.12 (d, C-5‘ose’), 71.58 (d, C-2‘ose’), 98.33 (d, C-1‘ose’), 117.32 (d, Ar—Cₘₑₜₐ), 121.51 (s, Ar—Cₘₑₛₒ), 122.10 (s, Ph-Cₘₑₛₒ), 122.14 (s, Ph-Cₘₑₛₒ), 128.76 (d, Ph-Cₘₑₜₐ), 129.86 (d, Ph-Cₚₐᵣₐ), 136.32 (d, Ar—Cₒᵣₜₕₒ), 137.37 (d, Ph-Cₒᵣₜₕₒ), 144.88 (s, Ph-Cᵢₚₛₒ), 157.82 (s, Ar—C_{OMan}), 171.36 (s, C═O), 170.45 (s, C═0), 170.51 (s, C═0), 170.71 (s, C═0) ppm. ESI-HRMS: C₅₈H₄₉N₄O₁₀⁺ ([M+H]⁺): calculated 961.3448, found 961.3444. UV/vis (CH₂Cl₂): λₘₐₓ (log ε/dm³ mol⁻¹ cm⁻¹): 415 (5.51), 513 (4.30), 547 (4.00), 591 (3.84), 647 (3.66) nm.

### 1.2 Preparation of 5-[4-(2,3,4,6,2′,3′,6′-heptaacetyl-β-D-lactosyl)phenyl]-10,15,20-triphenylporphyrin

In a typical experiment, under argon atmosphere, Zn(II)-5-(4-hydroxyphenyl)-10,15,20-triphenylporphyrin (60 mg, 86 μmol) was dissolved in 10 ml dry dichloromethane and 0.5 ml dry acetonitrile. Then, 2,3,4,6,2′,3′,6′-heptaacetyl-α-D-lactose trichloroacetimidate (500 mg, 640 μmol) and BF₃-Et₂O (10 μl, 80 μmol) were added. After stirring for 2 hours, the mixture was transferred to a separatory funnel. The organic layer was washed with water (2×100 ml) and the solvent was evaporated under reduced pressure. To remove the zinc, the residue was dissolved in 20 ml tetrahydrofuran, and 0.6 ml of hydrochloric acid (25%) were added. After stirring for 10 minutes, water (100 ml) and dichloromethane (150 ml) were added. The organic layer was separated and washed with water (2×100 ml). After drying with Na₂SO₄, the solvent was evaporated under reduced pressure. Further purification was achieved by flash chromatography, using dichloromethane/methanol 99:1 as the eluent. The analytically pure product (58 mg, 54%) was obtained as a violet crystalline solid after recrystallization from dichloromethane/aqueous methanol.

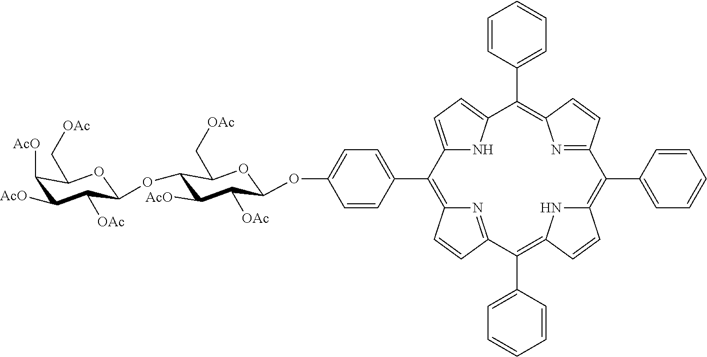

mp: >300° C., ¹H NMR (700 MHz, CDCl₃): δ=−2.80 (br s, 2H, NH), 1.99 (s, 3H, OAc), 2.09 (s, 3H, OAc), 2.11 (s, 3H, OAc), 2.13 (s, 3H, OAc), 2.15 (s, 3H, OAc), 2.19 (s, 3H, OAc), 2.21 (s, 3H, OAc), 3.96-4.04 (m, 3H, H-5′‘ose’, H-4′‘ose’, H-5‘ose’), 4.16 (dd, J=7.3, 11.2 Hz, 1H, H-6_{A}‘ose’), 4.22 (dd, J=6.3, 11.2 Hz, 1H, H-6_{B}‘ose’), 4.29 (dd, J=5.7, 11.9 Hz, 1H, H-6B′‘ose’), 4.58 (d, J=7.9 Hz, 1H, H-1 ‘ose’), 4.62 (dd, J=2.0, 11.9 Hz, 1H, H-6_{A}′‘ose’), 5.00 (dd, J=3.4, 10.4 Hz, 1H, H-3‘ose’), 5.18 (dd, J=8.0, 10.4 Hz, 1H, H-2‘ose’), 5.39-5.47 (m, 4H, H-4‘ose’, H-1 ‘ose’, H-2‘ose’, H-3‘ose’), 7.38 (d, J=8.5 Hz, 2H, Ar—Hₘₑta), 7.76-7.83 (m, 9H, 6×Ph-Hₘₑta, 3×Ph-Hₚₐᵣₐ), 8.15 (d, J=8.5 Hz, 2H, Ar—Hₒᵣₜₕₒ), 8.21-8.24 (m, 6H, Ph-Hₒᵣₜₕₒ), 8.81-8.84 (m, 8H, β-H) ppm. ¹³C NMR (176 MHz, CDCl₃): δ=20.55 (q, CH₃), 20.68 (q, CH₃), 20.69 (q, CH₃), 20.71 (q, CH₃), 20.87 (q, CH₃), 20.89 (q, CH₃), 60.90 (t, C-6‘ose’), 62.27 (t, C-6′‘ose’), 66.68 (d, C‘ose’), 69.17 (d, C-2‘ose’), 70.85 (d, C‘ose’), 71.02 (d, C-3‘ose’), 71.70 (d, C‘ose’), 72.97 (d, C‘ose’), 73.10 (d, C‘ose’), 76.45 (d, C‘ose’), 98.95 (d, C-1′‘ose’), 101.25 (d, C-1‘ose’), 115.05 (d, Ar—Cₘₑₜₐ), 119.17 (s, Ar—Cₘₑₛₒ), 120.20 (s, Ph-Cₘₑₛₒ), 120.22 (s, Ph-Cₘₑₛₒ), 126.70 (d, Ph-Cₘₑₜₐ), 127.75 (d, Ph-Cₚₐᵣₐ), 134.56 (d, Ph-Cₒᵣₜₕₒ), 135.56 (d, Ar—Cₒᵣₜₕₒ), 137.25 (s, Ar—Cᵢₚₛₒ), 142.14 (s, Ph-Cᵢₚₛₒ), 156.64 (s, Ar—C_{OLac}), 169.15 (s, C═0), 169.78 (s, C═0), 169.84 (s, C═0), 170.08 (s, C═0), 170.17 (s, C═0), 170.37 (s, C═0), 170.40 (s, C═0) ppm. ESI-HRMS: C₇₀H₆₅N₄O₁₈⁺ ([M+H]⁺): calculated 1249.4294, found 1249.4248. UV/vis (CH₂Cl₂): Aₘₐₓ (log E/dm³ mol⁻¹ cm⁻¹): 418 (5.56), 515 (4.26), 550 (3.94), 590 (3.81), 647 (370) nm.

### 1.3 Preparation of 5-[4-(2,3,4,6-tetraacetyl-β-D-glucosyl)phenyl]-10,15,20-tris-[3,5-bis-(trifluoro-methyl)phenyl]-porphyrin

In a typical experiment, under argon atmosphere, Zn(II)-5-(4-hydroxyphenyl)-10,15,20-tris-[3,5-bis-(trifluoromethyl)phenyl]-porphyrin (50 mg, 43 μmol) was dissolved in 10 ml dry dichloromethane. Then, 2,3,4,6-tetraacetyl-D-glucose trichloroacetimidate (40 mg, 81 μmol) and BF₃-Et₂O (20 μl, 0.2 mmol) were added. After stirring for 20 minutes, the mixture was transferred to a separatory funnel. The organic layer was washed with water (2×50 ml) and the solvent was evaporated under reduced pressure. To remove the zinc, the residue was dissolved in 10 ml tetrahydrofuran, and 1.0 ml of hydrochloric acid (25%) were added. After stirring for 10 minutes, water (50 ml) and dichloromethane (75 ml) were added. The organic layer was separated and washed with water (2×50 ml). After drying with Na₂SO₄, the solvent was evaporated under reduced pressure. Further purification was achieved by flash chromatography, using dichloromethane/ethyl acetate 95:5 as the eluent. The analytically pure product (49 mg, 83%) was obtained as a violet crystalline solid after recrystallization from dichloromethane/aqueous methanol.

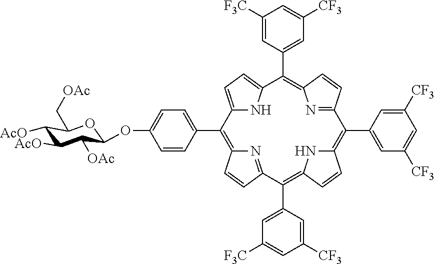

mp: 154° C., ¹H NMR (500 MHz, CDCl₃): δ=−2.84 (br s, 2H, NH), 2.11 (s, 3H, OAc), 2.12 (s, 3H, OAc), 2.13 (s, 3H, OAc), 2.23 (s, 3H, OAc), 4.08 (ddd, J=2.5, 5.3, 10.0 Hz, 1H, H-5‘ose’), 4.33 (dd, J=2.5, 12.4 Hz, 1H, H-6_{A}‘ose’), 4.43 (dd, J=5.3, 12.4 Hz, 1H, H-6_{B}‘ose’), 5.33 (dd, J=9.5, 10.0 Hz, 1H, H-4‘ose’), 5.47 (dd, J=9.5, 9.5 Hz, 1H, H-3‘ose’), 5.49 (d, J=7.7 Hz, 1H, H-1 ‘ose’), 5.50-5.53 (m, 1H, H-2‘ose’), 7.44 (d, J=8.5 Hz, 2H, 2×Ar-Hₘₑₜₐ), 8.16 (d, J=8.5 Hz, 2H, 2×Ar-Hₒᵣₜₕₒ), 8.38 (br s, 3H, 3×Ar_{F}—Hₚₐᵣₐ), 8.70 (br s, 6H, 6×Ar_{F}—Hₒᵣₜₕₒ), 8.75 (d, J=4.8 Hz, 2H, β-H), 8.79 (s, 4H, β-H), 8.99 (d, J=4.8 Hz, 2H, β-H) ppm. ¹³C NMR (126 MHz, CDCl₃): δ=20.74 (q, OCH₃), 20.78 (q, OCH₃), 20.86 (q, OCH₃), 20.91 (q, OCH₃), 62.16 (t, C-6‘ose’), 68.43 (d, C-4‘ose’), 71.39 (d, C-2‘ose’), 72.44 (d, C-5‘ose’), 72.88 (d, C-3‘ose’), 99.19 (d, C-1‘ose’), 115.40 (d, Ar—Cₘₑₜₐ), 116.77 (s, Ar_{F}—Cₘₑₛₒ), 117.19 (s, Ar_{F}—Cₘₑₛₒ), 120.31 (s, Ar_{F}—Cₘₑₛₒ), 121.59 (s, Ar—Cₘₑₛₒ), 122.27 (d, Ar_{F}—Cₚₐᵣₐ), 122.48, 124.65, 126.82, 130.62 (q, CF₃), 130.65 (q, CF₃), 133.77 (d, Ar_{F}—Cₒᵣₜₕₒ), 133.80 (d, Ar_{F}—Cₒᵣₜₕₒ), 135.75 (d, Ar—Cₒᵣₜₕₒ), 136.37 (s, Ar—Cᵢₚₛₒ), 143.84 (s, Ar_{F}—Cᵢₚₛₒ), 143.91 (s, Ar_{F}—Cᵢₚₛₒ), 157.05 (s, Ar—C_{OGlu}), 169.56 (s, C═0), 170.44 (s, C═0), 169.56 (s, C═0) ppm. ¹⁹F-NMR (471 MHz, CDCl₃): δ=−62.26 (s, 18 F, 6×CF₃) ppm. ESI-HRMS: C₆₄H₄₃F₁₈N₄O₁₀⁺ ([M+H]⁺): calculated 1369.2692, found 1369.2683. UV/vis (CH₂Cl₂): λₘₐₓ (log E/dm³ mol⁻¹ cm⁻¹): 420 (5.50), 515 (3.93), 554 (4.53), 593 (4.20), 645 (3.84) nm.

### 1.4 Preparation of 5,15-bis-[3-(2,3,4,6-tetraacetyl-β-D-glucosyl)-phenyl]-10,20-dihexylporphyrin

In a typical experiment, under argon atmosphere, Zn(II)-5,15-bis-(3-hydroxyphenyl)-10,20-dihexylporphyrin (75 mg, 0.10 mmol) was dissolved in 20 ml dry dichloromethane and 0.5 ml dry acetonitrile. Then, 2,3,4,6-tetraacetyl-D-glucose trichloroacetimidate (250 mg, 507 μmol) and BF₃-Et₂O (5.0 μl, 40 μmol) were added. After stirring for 2 hours, the mixture was transferred to a separatory funnel. The organic layer was washed with water (2×50 ml) and the solvent was evaporated under reduced pressure. To remove the zinc, the residue was dissolved in 20 ml tetrahydrofuran, and 1.0 ml of hydrochloric acid (25%) were added. After stirring for 10 minutes, water (50 ml) and dichloromethane (75 ml) were added. The organic layer was separated and washed with water (2×50 ml). After drying with Na₂SO₄, the solvent was evaporated under reduced pressure. Further purification was achieved by flash chromatography, using dichloromethane/ethyl acetate 95:5 as the eluent. The analytically pure product (108 mg, 78%) was obtained as a violet crystalline solid after recrystallization from dichloromethane/aqueous methanol.

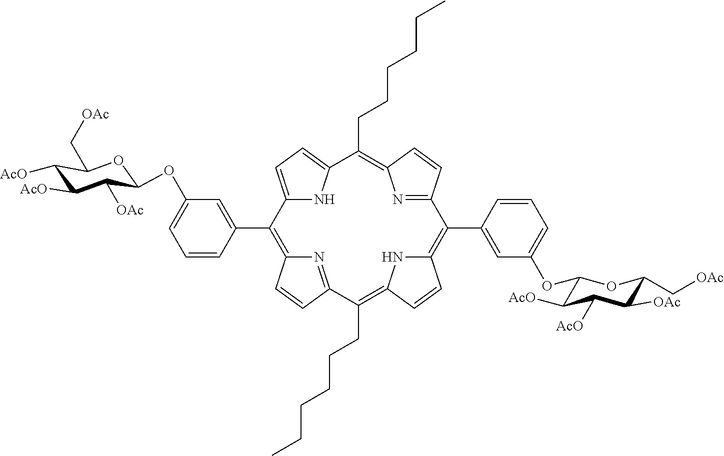

mp: 205° C., ¹H NMR (500 MHz, CDCl₃): δ=−2.72 (m, 2H, NH), 0.91-0.96 (m, 6H, 2×CH₃), 1.31 (s, 3H, OAc), 1.32 (s, 3H, OAc), 1.36-1.43 (m, 4H, 2×CH₂), 1.48-1.57 (m, 4H, 2×CH₂), 1.77-1.84 (m, 4H, 2×CH₂), 1.98 (s, 6H, 2 xOAc), 2.04 (s, 6H, 2×OAc), 2.11 (s, 6H, 2×OAc), 2.47-2.57 (m, 4H, 2×CH₂), 3.76-3.80 (m, 2H, H-5‘ose’), 4.03-4.07 (m, 2H, H-6_{A}‘ose’), 4.14-4.19 (m, 2H, H-6_{B}‘ose’), 4.93-5.00 (m, 4H, 2×CH₂), 5.18 (dd, J=9.3, 9.3 Hz, 2H, H-4‘ose’), 5.32 (dd, J=9.3, 9.3 Hz, 2H, H-3‘ose’), 5.36 (d, J=7.9 Hz, 2H, H-1‘ose’), 5.41 (dd, J=7.9, 9.3 Hz, 2H, H-2‘ose’), 7.43-7.46 (m, 2H, Ar—H), 7.65-7.69 (m, 2H, Ar—H), 7.84-7.87 (m, 2H, Ar—H), 7.92-7.95 (m, 2H, Ar—H), 8.89 (d, J=4.7 Hz, 4H, β-H), 9.42-9.46 (m, 4H, β-H) ppm. ¹³C NMR (126 MHz, CDCl₃): δ=14.22 (q, CH₃), 19.96 (q, OCH₃), 20.60 (q, OCH₃), 20.69 (q, OCH₃), 20.80 (q, OCH₃), 22.80 (t, CH₂), 30.32 (t, CH₂), 31.99 (t, CH₂), 35.42 (t, CH₂), 38.87 (t, CH₂), 61.98 (t, C-6‘ose’), 68.38 (d, C-4‘ose’), 71.39 (d, C-2‘ose’), 72.26 (d, C-5‘ose’), 72.89 (d, C-3‘ose’), 99.38 (d, C-1‘ose’), 116.71 (d, Ar—C), 118.10 (s, Ar—Cₘₑₛₒ), 120.19 (s, Ar—Cₘₑₛₒ), 122.78 (d, Ar—C), 127.67 (d, Ar—C), 129.95 (d, Ar—C), 144.32 (s, Ar—Cᵢₚₛₒ), 155.35 (s, Ar—C_{OGlu}), 169.45 (C═0), 170.31 (C═0), 170.46 (C═0) ppm. ESI-HRMS: C₇₂H₈₃N₄O₂₀⁺ ([M+H]⁺): calculated 1323.5601, found 1323.5578. UV/vis (CH₂Cl₂): Aₘₐₓ (log ε/dm³ mol⁻¹ cm⁻¹): 419 (5.59), 518 (4.28), 554 (4.03), 597 (3.71), 653 (3.81) nm.

### 1.5 Preparation of 5,15-bis-[4-(2,3,4,6-tetraacetyl-β-D-glucosyl)-phenyl]-10,20-dihexylporphyrin

In a typical experiment, under argon atmosphere, Zn(II)-5,15-bis-(4-hydroxyphenyl)-10,20-dihexylporphyrin (75 mg, 0.10 mmol) was dissolved in 20 ml dry dichloromethane and 0.5 ml dry acetonitrile. Then, 2,3,4,6-tetraacetyl-D-glucose trichloroacetimidate (250 mg, 507 μmol) and BF₃-Et₂O (5.0 μl, 40 μmol) were added. After stirring for 2 hours, the mixture was transferred to a separatory funnel. The organic layer was washed with water (2×50 ml) and the solvent was evaporated under reduced pressure. To remove the zinc, the residue was dissolved in 20 ml tetrahydrofuran, and 1.0 ml of hydrochloric acid (25%) were added. After stirring for 10 minutes, water (50 ml) and dichloromethane (75 ml) were added. The organic layer was separated and washed with water (2×50 ml). After drying with Na₂SO₄, the solvent was evaporated under reduced pressure. Further purification was achieved by flash chromatography, using dichloromethane/ethyl acetate 95:5 as the eluent. The analytically pure product (113 mg, 82%) was obtained as a violet crystalline solid after recrystallization from dichloromethane/aqueous methanol.

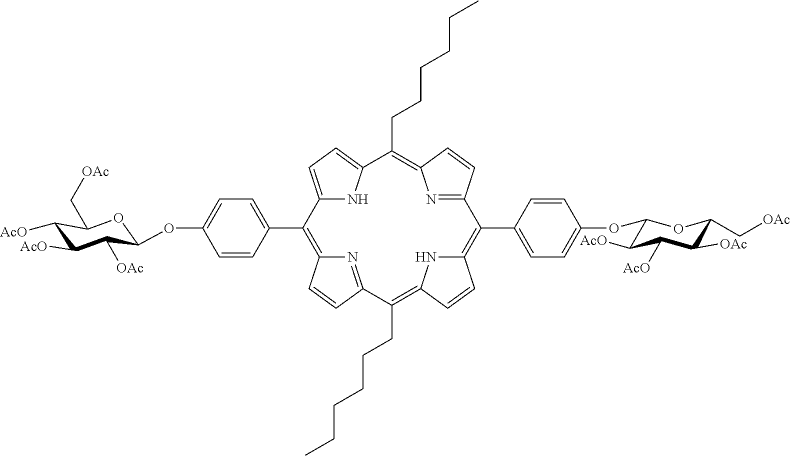

mp: 204° C., ¹H NMR (500 MHz, CDCl₃): δ=−2.70 (br s, 2H, NH), 0.91 (t, J=7.5 Hz, 6H, 2×CH₃), 1.33-1.41 (m, 4H, 2×CH₂), 1.46-1.52 (m, 4H, 2×CH₂), 1.75-1.81 (m, 4H, 2×CH₂), 2.11 (s, 6H, 2×OAc), 2.12 (s, 6H, 2×OAc), 2.14 (s, 6H, 2×OAc), 2.24 (s, 6H, 2×OAc), 2.47-2.53 (m, 4H, 2×CH₂), 4.07 (ddd, J=2.4, 5.5, 10.2 Hz, 2H, 2×H-5‘ose’), 4.32 (dd, J=2.4, 12.3 Hz, 2H, 2×H-6_{A}‘ose’), 4.44 (dd, J=5.5, 12.3 Hz, 2H, 2×H-6_{B}‘ose’), 4.92-4.97 (m, 4H, 2×CH₂), 5.32 (dd, J=9.4, 10.1 Hz, 2H, 2×H-4‘ose’), 5.45-5.53 (m, 6H, 2×H-1 ‘ose’, 2×H-2‘ose’, 2×H-3‘ose’), 7.38 (d, J=8.5 Hz, 4H, Ar—Hₘₑₜₐ), 8.11 (d, J=8.5 Hz, 4H, Ar—Hₒᵣₜₕₒ), 8.85 (d, J=4.8 Hz, 4H, β-H), 9.42 (d, J=4.8 Hz, 4H, β-H) ppm. ¹³C NMR (126 MHz, CDCl₃): δ=14.11 (q, CH₃), 20.64 (q, OCH₃), 20.72 (q, OCH₃), 20.82 (q, OCH₃), 22.69 (t, CH₂), 30.32 (t, CH₂), 31.89 (t, CH₂), 35.31 (t, CH₂), 38.68 (t, CH₂), 62.14 (t, C-6‘ose’), 68.46 (d, C-4‘ose’), 71.38 (d, C-2‘ose’), 72.32 (d, C-5‘ose’), 72.89 (d, C-3‘ose’), 99.30 (d, C-1‘ose’), 114.93 (d, Ar—Cₘₑₜₐ), 117.96 (s, Ar—Cₘₑₛₒ), 119.93 (s, Ar—Cₘₑₛₒ), 135.40 (d, Ar—Cₒᵣₜₕₒ), 137.79 (s, Ar—Cᵢₚₛₒ), 156.57 (s, Ar—C_{OGlu}), 169.48 (s, C═0), 170.35 (s, C═0), 170.65 (s, C═0) ppm. ESI-HRMS: C₇₂H₈₃N₄O₂₀⁺ ([M+H]⁺): calculated 1323.5601, found 1323.5572. UV/vis (CH₂Cl₂): λₘₐₓ (log ε/dm³ mol⁻¹ cm⁻¹): 419 (5.59), 518 (4.28), 554 (4.03), 597 (3.71), 653 (3.81) nm.

### 1.6 Preparation of 5,15-bis-[4-(2,3,4,6-tetraacetyl-β-D-galactosyl)-phenyl]-10,20-dihexylporphyrin

In a typical experiment, under argon atmosphere, Zn(II)-5,15-bis-(4-hydroxyphenyl)-10,20-dihexylporphyrin (50 mg, 69 μmol) was dissolved in 15 ml dry dichloromethane and 0.3 ml dry acetonitrile. Then, 2,3,4,6-tetraacetyl-D-galactose trichloroacetimidate (165 mg, 335 μmol) and BF₃-Et₂O (5.0 μl, 40 μmol) were added. After stirring for 2 hours, the mixture was transferred to a separatory funnel. The organic layer was washed with water (2×50 ml) and the solvent was evaporated under reduced pressure. To remove the zinc, the residue was dissolved in 10 ml tetrahydrofuran, and 0.7 ml of hydrochloric acid (25%) were added. After stirring for 10 minutes, water (50 ml) and dichloromethane (75 ml) were added. The organic layer was separated and washed with water (2×50 ml). After drying with Na₂SO₄, the solvent was evaporated under reduced pressure. Further purification was achieved by flash chromatography, using dichloromethane/ethyl acetate 95:5 as the eluent. The analytically pure product (73 mg, 80%) was obtained as a violet crystalline solid after recrystallization from dichloromethane/aqueous methanol.

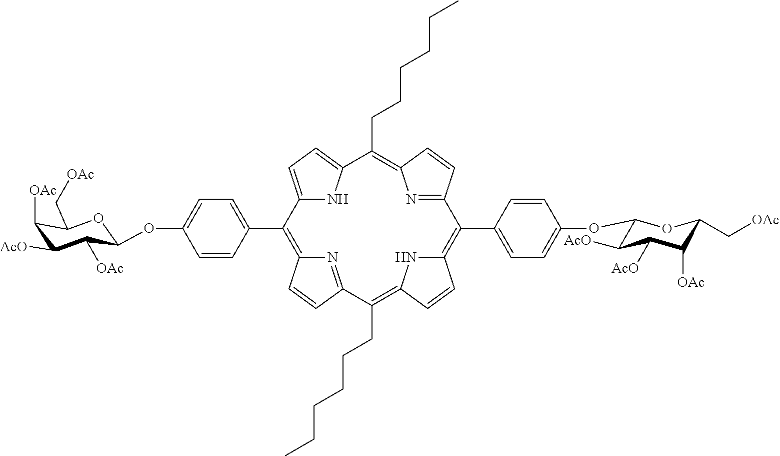

mp: 161° C., ¹H NMR (500 MHz, CDCl₃): δ=−2.70 (br s, 2H, NH), 0.91 (t, J=7.4 Hz, 6H, 2×CH₃), 1.34-1.41 (m, 4H, 2×CH₂), 1.46-1.54 (m, 4H, 2×CH₂), 1.76-1.82 (m, 4H, 2×CH₂), 2.07 (s, 6H, 2×OAc), 2.08 (s, 6H, 2 xOAc), 2.24 (s, 6H, 2×OAc), 2.27 (s, 6H, 2×OAc), 2.47-2.53 (m, 4H, 2×CH₂), 4.22-4.26 (m, 2H, 2×H-5‘ose’), 4.29-4.39 (m, 4H, 2×H-6‘ose’), 4.94-4.96 (m, 4H, 2×CH₂), 5.28 (dd, J=3.6, 10.0 Hz, 2H, 2×H-3‘ose’), 5.41 (d, J=8.2 Hz, 2H, 2×H-1‘ose’), 5.57 (dd, J=0.9, 3.6 Hz, 2H, 2×H-4‘ose’), 5.71 (dd, J=8.2, 10.0 Hz, 2H, 2×H-2‘ose’), 7.40 (d, J=8.4 Hz, 4H, Ar—Hₘₑta), 8.12 (d, J=8.4 Hz, 4H, Ar—Hₒᵣₜₕₒ), 8.86 (d, J=4.8 Hz, 4H, β-H), 9.42 (d, J=4.8 Hz, 4H, β-H) ppm. ¹³C NMR (126 MHz, CDCl₃): δ=14.20 (q, CH₃), 20.74 (q, OCH₃), 20.79 (q, OCH₃), 20.83 (q, OCH₃), 21.02 (q, OCH₃), 22.78 (t, CH₂), 30.29 (t, CH₂), 31.98 (t, CH₂), 35.40 (t, CH₂), 38.78 (t, CH₂), 61.63 (t, C-6‘ose’), 67.13 (d, C-4‘ose’), 68.96 (d, C-2‘ose’), 71.10 (d, C-3‘ose’), 71.41 (d, C-5‘ose’), 99.94 (d, C-1‘ose’), 115.05 (d, Ar—Cₘₑₜₐ), 118.08 (s, Ar—Cₘₑₛₒ), 120.01 (s, Ar—Cₘₑₛₒ), 135.48 (d, Ar—Cₒᵣₜₕₒ), 137.85 (s, Ar—Cᵢₚₛₒ), 156.70 (s, Ar—C_{OGal}), 169.63 (s, C═0), 170.30 (s, C═0), 170.40 (s, C═0), 170.50 (s, C═0) ppm. ESI-HRMS: C₇₂H₈₃N₄O₂₀⁺ ([M+H]⁺): calculated 1323.5601, found 1323.5558. UV/vis (CH₂Cl₂): λₘₐₓ (log ε/dm³ mol⁻¹ cm⁻¹): 419 (5.60), 519 (4.26), 554 (4.02), 597 (3.69), 654 (3.80) nm.

### 1.7 Preparation of 5,15-bis-[3-(2,3,4,6-tetraacetyl-β-D-glucosyl)-phenyl]-10,20-diphenylporphyrin

In a typical experiment, under argon atmosphere, Zn(II)-5,15-bis-(3-hydroxyphenyl)-10,20-diphenylporphyrin (20 mg, 28 μmol) was dissolved in 4 ml dry dichloromethane and 0.2 ml dry acetonitrile. Then, 2,3,4,6-tetraacetyl-D-glucose trichloroacetimidate (50 mg, 102 μmol) and BF₃-Et₂O (1.0 μl, 8.0 μmol) were added. After stirring for 2 hours, the mixture was transferred to a separatory funnel. The organic layer was washed with water (2×50 ml) and the solvent was evaporated under reduced pressure. To remove the zinc, the residue was dissolved in 4 ml tetrahydrofuran, and 0.2 ml of hydrochloric acid (25%) were added. After stirring for 10 minutes, water (50 ml) and dichloromethane (75 ml) were added. The organic layer was separated and washed with water (2×50 ml). After drying with Na₂SO₄, the solvent was evaporated under reduced pressure. Further purification was achieved by flash chromatography, using dichloromethane/ethyl acetate 95:5 as the eluent. The analytically pure product (28 mg, 75%) was obtained as a violet crystalline solid after recrystallization from dichloromethane/aqueous methanol.

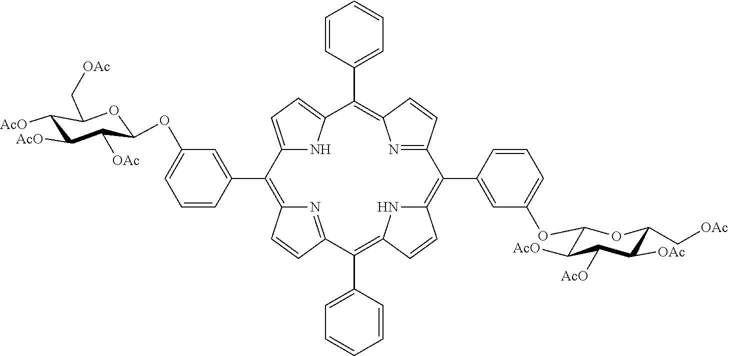

mp: >300° C., ¹H NMR (500 MHz, CDCl₃): δ=−2.83 (br s, 2H, NH), 1.31-1.34 (m, 6H, 2×OAc), 1.97-1.98 (m, 6H, 2×OAc), 2.03 (m, 6H, 2×OAc), 2.09 (s, 6H, 2×OAc), 3.76-3.80 (m, 2H, H-5‘ose’), 4.00-4.04 (m, 2H, H-6_{A}‘ose’), 4.13-4.17 (m, 2H, H-6_{B}‘ose’), 5.13-5.18 (m, 2H, H-4‘ose’), 5.29-5.40 (m, 6H, H-1‘ose’, H-2‘ose’, H-3‘ose’), 7.41-7.44 (m, 2H, Ar—H), 7.64-7.69 (m, 2H, Ar—H), 7.75-7.79 (m, 6H, 4×Ph-Hₘₑta, 2×Ph-Hₚₐᵣₐ), 7.85-7.88 (m, 2H, Ar—H), 7.93-7.96 (m, 2H, Ar—H), 8.16-8.23 (m, 4H, Ph-Hₒᵣₜₕₒ), 8.84-8.88 (m, 8H, β-H) ppm. ¹³C NMR (126 MHz, CDCl₃): δ=19.99 (q, CH₃), 20.63 (q, CH₃), 20.70 (q, CH₃), 20.81 (q, CH₃), 61.96 (t, C-6‘ose’), 68.30 (d, C-4‘ose’), 71.30 (d, C-2‘ose’), 72.23 (d, C-5‘ose’), 72.81 (d, C-3‘ose’), 99.31 (d, C-1‘ose’), 116.71 (d, Ar—C), 119.31 (s, Ph-Cₘₑₛₒ), 120.50 (s, Ar—Cₘₑₛₒ), 122.84 (d, Ar—C), 126.88 (d, Ph-Cₘₑta), 127.84 (d, Ar—C), 127.95 (d, Ph-Cₚₐᵣₐ), 129.99 (d, Ar—C), 134.61 (d, Ph-Cₒᵣₜₕₒ), 142.01 (s, Ph-Cᵢₚₛₒ), 143.78 (s, Ar—Cᵢₚₛₒ), 155.41 (s, Ar—C_{OGlu}), 169.46 (s, C═0), 170.32 (s, C═0), 170.48 (s, C═0) ppm. ESI-HRMS: C₇₂H₆₆N₄O₂₀Na⁺ ([M+Na]⁺): calculated 1329.4120, found 1329.4168. UV/vis (CH₂Cl₂): λₘₐₓ (log ε/dm³ mol⁻¹ cm⁻¹): 415 (5.41), 513 (3.79), 547 (3.56), 591 (3.26), 648 (3.36) nm.

### 1.8 Preparation of 5,10,15,20-tetrakis-[4-(2,3,4,6-tetraacetyl-β-D-glucosyl)phenyl]-porphyrin (Reference example)

In a typical experiment, under argon atmosphere, Zn(II)-5,10,15,20-(4-hydroxyphenyl)-porphyrin (50 mg, 62 μmol) was dissolved in 8 ml dry dichloromethane, 1 ml tetrahydrofuran and 1 ml acetonitrile. Then, 2,3,4,6-tetraacetyl-D-glucose trichloroacetimidate (0.8 g, 1.5 mmol) and BF₃-Et₂O (4.0 μl, 32 μmol) were added. After stirring for 3 hours, the mixture was transferred to a separatory funnel. The organic layer was washed with water (2×50 ml) and the solvent was evaporated under reduced pressure. To remove the zinc, the residue was dissolved in 10 ml tetrahydrofuran, and 0.8 ml of hydrochloric acid (25%) were added. After stirring for 10 minutes, water (50 ml) and dichloromethane (75 ml) were added. The organic layer was separated and washed with water (2×50 ml). After drying with Na₂SO₄, the solvent was evaporated under reduced pressure. Further purification was achieved by flash chromatography, using dichloromethane/methanol 99:1 as the eluent. The analytically pure product (93 mg, 69%) was obtained as a violet crystalline solid after recrystallization from dichloromethane/aqueous methanol.

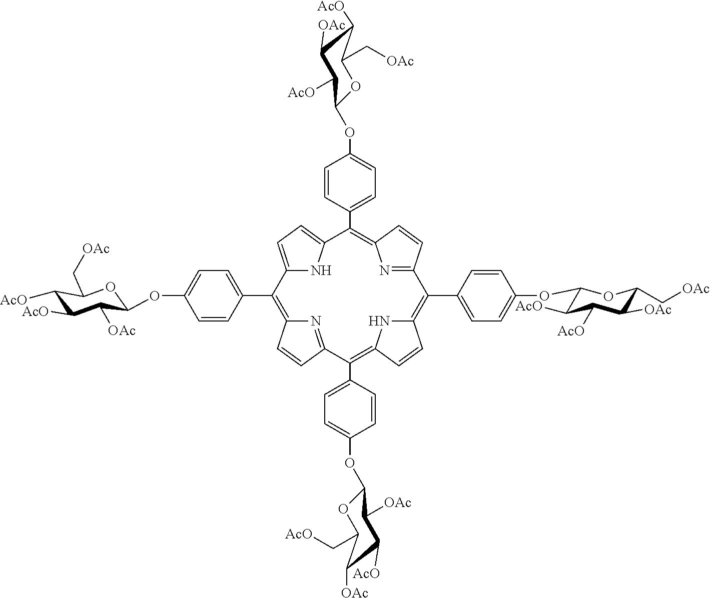

mp: 287° C., ¹H NMR (500 MHz, CDCl₃): δ=−2.83 (br s, 2H, NH), 2.10 (s, 12H, 4×OAc), 2.11 (s, 12H, 4×OAc), 2.12 (s, 12H, 4×OAc), 2.21 (s, 12H, 4×OAc), 4.06 (ddd, J=2.5, 5.4, 10.1 Hz, 4H, H-5‘ose’), 4.30 (dd, J=2.5, 12.3 Hz, 4H, H-6_{A}‘ose’), 4.42 (dd, J=5.4, 12.3 Hz, 4H, H-6_{B}‘ose’), 5.31 (dd, J=9.6, 10.1 Hz, 4H, H-4‘ose’), 5.45 (dd, J=9.6, 9.6 Hz, 4H, H-3‘ose’), 5.47 (d, J=7.6 Hz, 4H, H-1‘ose’), 5.49 (dd, J=7.6, 9.6 Hz, 4H, H-2‘ose’), 7.38 (d, J=8.5 Hz, 8H, 8×Ar-Hₘₑₜₐ), 8.12 (d, J=8.5 Hz, 8H, 8 xAr-Hₒᵣₜₕₒ), 8.85 (s, 8H, β-H) ppm. ¹³C NMR (126 MHz, CDCl₃): δ=20.73 (q, CH₃), 20.77 (q, CH₃), 20.86 (q, CH₃), 20.90 (q, CH₃), 62.18 (t, C-6‘ose’), 68.48 (d, C-4‘ose’), 71.43 (d, C-2‘ose’), 72.39 (d, C-5‘ose’), 72.93 (d, C-3‘ose’), 99.25 (d, C-1‘ose’), 115.17 (d, Ar—Cₘₑₜₐ), 119.43 (s, Ar—Cₘₑₛₒ), 135.62 (d, Ar—Cₒᵣₜₕₒ), 137.21 (s, Ar—Cᵢₚₛₒ), 156.74 (s, Ar—C_{OGlu}), 169.55 (s, C═0), 170.41 (s, C═0), 170.70 (s, C═0) ppm. ESI-HRMS: C₁₀₀H₁₀₃N₄O₄₀⁺ [M+H]⁺: calculated 1999.6149, found 1999.6078. UV/vis (CH₂Cl₂): λₘₐₓ (log ε/dm³ mol⁻¹ cm⁻¹): 420 (5.20), 515 (3.89), 551 (3.66), 594 (3.46), 649 (3.39) nm.

### 1.9 Preparation of 5,10,15,20-tetrakis-[3-(2,3,4,6-tetraacetyl-β-D-galactosyl)-phenyl]-porphyrin (Reference Example)

In a typical experiment, under argon atmosphere, Zn(II)-5,10,15,20-(3-hydroxyphenyl)-porphyrin (50 mg, 62 μmol) was dissolved in 8 ml dry dichloromethane, 1 ml tetrahydrofuran and 1 ml acetonitrile. Then, 2,3,4,6-tetraacetyl-D-galactose trichloroacetimidate (0.8 g, 1.5 mmol) and BF₃-Et₂O (3.5 μl, 28 μmol) were added. After stirring for 3 hours, the mixture was transferred to a separatory funnel. The organic layer was washed with water (2×50 ml) and the solvent was evaporated under reduced pressure. To remove the zinc, the residue was dissolved in 8 ml tetrahydrofuran, and 0.4 ml of hydrochloric acid (25%) were added. After stirring for 10 minutes, water (50 ml) and dichloromethane (75 ml) were added. The organic layer was separated and washed with water (2×50 ml). After drying with Na₂SO₄, the solvent was evaporated under reduced pressure. Further purification was achieved by flash chromatography, using dichloromethane/methanol 99:1 as the eluent. The analytically pure product (97 mg, 78%) was obtained as a violet crystalline solid after recrystallization from dichloromethane/aqueous methanol.

This porphyrin is an atropisomer.

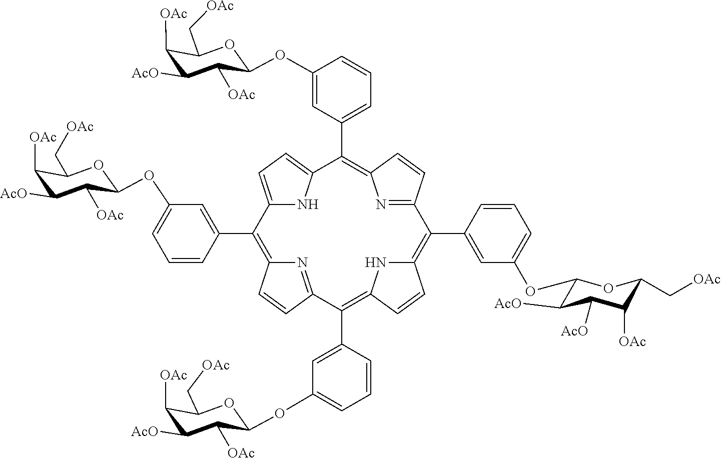

mp: 225° C., ¹H NMR (500 MHz, (CD₃)₂CO): δ=−2.84 (br s, 2H, NH), 0.97-1.04 (m, 12H, 4×OAc), 1.90-1.93 (m, 12H, 4×OAc), 2.06-2.09 (m, 12H, 4×OAc), 2.12 (s, 12H, 4×OAc), 4.00-4.09 (m, 8H, H-6‘ose’), 4.32-4.37 (m, 4H, H-5‘ose’), 5.24-5.28 (m, 4H, H-3‘ose’), 5.37-5.40 (m, 4H, H-4‘ose’), 5.50-5.55 (m, 4H, H-2‘ose’), 5.71-5.75 (m, 4H, H-1‘ose’), 7.53-7.57 (m, 4H, Ar—H), 7.75-7.80 (m, 4H, Ar—H), 7.92-8.06 (m, 8H, Ar—H), 8.93-8.97 (m, 8H, β-H) ppm. ESI-HRMS: C₁₀₀H₁₀₃N₄O₄₀⁺ ([M+H]⁺): calculated 1999.6149, found 1999.6140. UV/vis ((CH₃)₂CO): λₘₐₓ (log ε/dm³ mol⁻¹ cm⁻¹): 418 (5.54), 513 (4.22), 548 (3.74), 589 (3.69), 645 (3.38) nm.

### 1.10 Preparation of 5-[3-(2,3,4,6-tetraacetyl-β-D-glucosyl)phenyl]-10,15,20-tris-(pentafluorophenyl)-porphyrin

In a typical experiment, under argon atmosphere, Zn(II)-[5-(3-hydroxyphenyl)-10,15,20-tris-(pentafluorophenyl)-porphyrin (60 mg, 62 μmol) was dissolved in 10 ml dry dichloromethane. Then, 2,3,4,6-tetraacetyl-D-glucose trichloroacetimidate (132 mg, 268 μmol) and BF₃-Et₂O (2.8 μl, 22 μmol) were added. After stirring for 20 minutes, the mixture was transferred to a separatory funnel. The organic layer was washed with water (2×50 ml) and the solvent was evaporated under reduced pressure. To remove the zinc, the residue was dissolved in 10 ml tetrahydrofuran, and 1.5 ml of hydrochloric acid (25%) were added; this step was repeated two more times. Then water (50 ml) and dichloromethane (75 ml) were added. The organic layer was separated and washed with water (2×50 ml). After drying with Na₂SO₄, the solvent was evaporated under reduced pressure. Further purification was achieved by flash chromatography, using dichloromethane/ethyl acetate 98:2 as the eluent. The analytically pure product (32 mg, 44%) was obtained as a violet crystalline solid after recrystallization from dichloromethane/aqueous methanol.

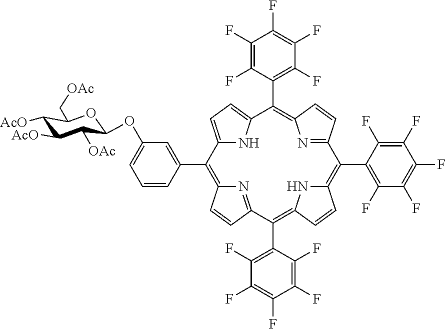

mp: 190° C., ¹H NMR (700 MHz, CDCl₃): δ=−2.84 (m, 2H, NH), 1.41 (s, 3H, OAc), 2.01 (s, 3H, OAc), 2.06 (s, 3H, OAc), 2.13 (s, 3H, OAc), 3.83 (ddd, J=2.4, 5.6, 10.1 Hz, 1H, H-5‘ose’), 4.11 (dd, J=2.4, 12.2 Hz, 1H, H-6_{A}‘ose’), 4.18 (dd, J=5.6, 12.2 Hz, 1H, H-6_{B}‘ose’), 5.21 (dd, J=9.2, 10.1 Hz, 1H, H-4‘ose’), 5.35 (dd, J=9.2, 9.2 Hz, H-3‘ose’), 5.39 (d, J=7.7 Hz, 1H, H-1‘ose’), 5.42 (dd, J=7.7, 9.2 Hz, 1H, H-2‘ose’), 7.49-7.51 (m, 1H, Ar—H), 7.73-7.76 (m, 1H, Ar—H), 7.88-7.92 (m, 1H, Ar—H), 7.95-7.98 (m, 1H, Ar—H), 8.86-8.88 (m, 2H, β-H), 8.91-8.94 (m, 4H, β-H), 9.01-9.03 (m, 2H, β-H) ppm. ¹³C NMR (176 MHz, CDCl₃): δ=19.87 (OCH₃), 20.53 (OCH₃), 20.60 (OCH₃), 20.72 (OCH₃), 61.94 (C-6‘ose’), 68.26 (C-4‘ose’), 71.23 (C-2‘ose’), 72.19 (C-5‘ose’), 72.70 (C-3‘ose’), 99.07 (C-1‘ose’), 102.07 (Ar_{F}—Cₘₑₛₒ), 103.11 (Ar_{F}—Cₘₑₛₒ), 115.60-115.94 (Ar_{F}—Cᵢₚₛₒ), 117.21 (Ar—C), 122.12 (Ar—Cₘₑₛₒ), 122.81 (Ar—C), 128.05 (Ar—C), 129.90 (Ar—C), 136.77-136.94 (Ar_{F}—C), 138.21-138.32 (Ar_{F}—C), 141.48-141.56 (Ar_{F}—C), 142.47 (Ar—Cᵢₚₛₒ), 142.86-142.95 (Ar_{F}—C), 145.76-145.96 (Ar_{F}—C), 147.12-147.35 (Ar_{F}—C), 155.33 (Ar—C_{OGlu}), 169.34 (C═0), 169.35 (C═0), 170.21 (C═0), 170.31 (C═0) ppm. ¹⁹F NMR (471 MHz, CDCl₃): δ=−161.54-−161.27 (m, 6 F, Ar—Fₘₑₜₐ), −151.57-−151.42 (m, 3 F, Ar—Fₚₐᵣₐ), −136.75-−136.39 (m, 6 F, Ar—Fₒᵣₜₕₒ) ppm. ESI-HRMS: C₅₈H₃₃F₁₅N₄O₁₀Na⁺ ([M+Na]⁺): calculated 1253.1849, found 1253.1855. UV/vis (CH₂Cl₂): λₘₐₓ (log ε/dm³ mol⁻¹ cm⁻¹): 414 (5.50), 508 (4.33), 585 (3.83) nm.

### Example 2—Preparation of Glycosylated Deacetylated Porphyrins

### 2.1 Preparation of 5-(4-α-D-mannosylphenyl)-10,15,20-triphenylporphyrin

In a typical experiment, under argon atmosphere, 5-[4-(2,3,4,6-tetraacetyl-α-D-mannosyl)phenyl]-10,15,20-triphenylporphyrin (40 mg, 42 μmol) was dissolved in 5.0 ml dry tetrahydrofuran and 5.0 ml methanol. Then a solution of sodium methanolate in dry methanol (1.5 ml, 0.06 N) was added. After 2 h, the solvent was evaporated under reduced pressure and the crude product was purified by flash chromatography, using dichloromethane/methanol 9:1 as the eluent. The desired product (32 mg, 98%) was obtained as a violet crystalline solid.

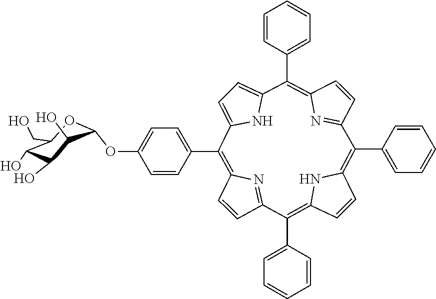

mp: 251° C., ¹H NMR (500 MHz, (CD₃)₂SO): δ=−2.91 (br s, 2H, NH), 3.60-3.66 (m, 2H, H-4‘ose’, H-6_{A}‘ose’), 3.68-3.72 (m, 1H, H-5‘ose’), 3.76-3.81 (m, 1H, H-6_{B}‘ose’), 3.86-3.90 (m, 1H, H-3‘ose’), 4.04-4.07 (m, 1H, H-2‘ose’), 4.63 (dd, J=5.9, 5.9 Hz, 1H, OH-6‘ose’), 4.87 (d, J=5.6 Hz, 1H, OH-3‘ose’), 4.95 (d, J=5.6 Hz, 1H, OH-4‘ose’), 5.17 (d, J=4.6 Hz, 1H, OH-2‘ose’), 5.72 (d, J=1.8 Hz, 1H, H-1‘ose’), 7.53 (d, J=8.7 Hz, 2H, Ar—Hₘₑₜₐ), 7.80-7.85 (m, 9H, 6×Ph-Hₘₑta, 3×Ph-Hₚₐᵣₐ), 8.13 (d, J=8.7 Hz, 2H, Ar—Hₒᵣₜₕₒ), 8.20-8.23 (m, 6H, Ph-Hₒᵣₜₕₒ), 8.82 (s, 6H, β-H), 8.88 (d, J=4.6 Hz, 2H, β-H) ppm. ¹³C NMR (126 MHz, (CD₃)₂SO): 6=61.79 (t, C-6‘ose’), 67.44 (d, C-4‘ose’), 70.84 (d, C-2‘ose’), 71.42 (d, C-3‘ose’), 75.89 (d, C-5‘ose’), 100.00 (d, C-‘ose’), 115.91 (d, Ar—Cₘₑₜₐ), 120.43 (s, Ar—Cₘₑₛₒ), 120.52 (s, Ar—Cₘₑₛₒ), 127.56 (d, Ph-Cₘₑₜₐ), 128.64 (d, Ph-Cₚₐᵣₐ), 134.78 (d, Ph-Cₒᵣₜₕₒ), 135.28 (s, Ar—Cᵢₚₛₒ), 135.92 (Ar—Cₒᵣₜₕₒ), 141.78 (s, Ph-Cᵢₚₛₒ), 157.15 (s, Ar—C_{OMan}) ppm. ESI-HRMS: C₅₀H₄₁N₄O₆⁺ ([M+H]⁺): calculated 793.3021, found 793.3067. UV/vis ((CH₃)₂SO): λₘₐₓ (log ε/dm³ mol⁻¹ cm⁻¹): 415 (5.38), 513 (4.10), 547 (3.77), 591 (4.01), 647 (3.40) nm.

### 2.2 Preparation of 5-(4-β-D-lactosylphenyl)-10,15,20-triphenylporphyrin

In a typical experiment, under argon atmosphere, 5-[4-(2,3,4,6,2′,3′,6′-heptaacetyl-β-D-lactosyl)phenyl]-10,15,20-triphenylporphyrin (37 mg, 30 μmol) was dissolved in 6.0 ml dry tetrahydrofuran and 6.0 ml methanol. Then a solution of sodium methanolate in dry methanol (2.0 ml, 0.06 N) was added. After 2 h, the solvent was evaporated under reduced pressure and the crude product was purified by flash chromatography, using dichloromethane/methanol 9:1 as the eluent. The desired product (28 mg, 98%) was obtained as a violet crystalline solid.

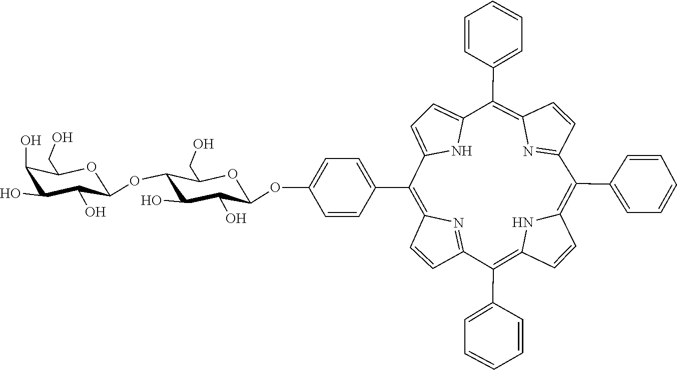

mp: >300° C., ¹H NMR (500 MHz, (CD₃)₂SO): δ=−2.93 (s, 2H, NH), 2.98-3.03 (m, 2H, H‘ose’), 3.48-3.79 (m, 9H, ‘ose’), 3.85-3.89 (m, 1H, ‘ose’), 4.35 (d, J=7.2 Hz, 1H, H‘ose’), 4.68 (d, J=4.6 Hz, 1H, OH‘ose’), 4.82-4.88 (m, 2H, OH‘ose’), 4.93 (d, J=1.6 Hz, 1H, ‘ose’), 4.96 (d, J=5.3 Hz, 1H, ‘ose’), 5.20 (d, J=4.0 Hz, 1H, ‘ose’), 5.33 (d, J=7.8 Hz, 1H, H‘ose’), 5.70 (d, J=5.3 Hz, 1H, OH‘ose’), 7.48 (d, J=8.6 Hz, 2H, Ar—Hₘₑₜₐ), 7.81-7.86 (m, 9H, 6×Ph-Hₘₑₜₐ, 3×Ph-Hₚₐᵣₐ), 8.13 (d, J=8.6 Hz, 2H, Ar—Hₒᵣₜₕₒ), 8.20-8.23 (m, 6H, Ph-Hₒᵣₜₕₒ), 8.81 (s, 6H, β-H), 8.88 (d, J=4.7 Hz, 2H, β-H) ppm. ¹³C NMR (126 MHz, (CD₃)₂SO): δ=60.62 (C‘ose’), 60.83 (C‘ose’), 68.61 (C‘ose’), 71.33 (C‘ose’), 73.77 (C‘ose’), 73.95 (C‘ose’), 75.45 (C‘ose’), 75.70 (C‘ose’), 76.13 (C‘ose’), 80.61 (C‘ose’), 104.34 (C‘ose’), 115.12 (d, Ar—Cₘₑₜₐ), 120.42 (s, Ar—Cₘₑₛₒ), 120.45 (s, Ph-Cₘₑₛₒ), 120.54 (s, Ph-Cₘₑₛₒ), 127.59 (d, Ph-Cₘₑₜₐ), 128.67 (d, Ph-Cₚₐᵣₐ), 134.78 (d, Ph-Coho), 135.81 (d, Ar—Cₒᵣₜₕₒ), 141.76 (s, Ph-Cᵢₚₛₒ), 157.91 (s, Ar—C_{OLac}) ppm. ESI-HRMS: C₅₆H₅₁N₄O₁₁⁺ ([M+H]⁺): calculated 955.3554, found 955.3554. UV/vis ((CH₃)₂SO): λₘₐₓ (log ε/dm³ mol⁻¹ cm⁻¹): 419 (4.79), 515 (3.54), 551 (3.27), 592 (3.10), 647 (3.05) nm.

### 2.3 Preparation of 5,15-bis-(3-β-D-glucosylphenyl)-10,20-dihexylporphyrin

In a typical experiment, under argon atmosphere, 5,15-bis-[3-(2,3,4,6-tetraacetyl-β-D-glucosyl)-phenyl]-10,20-dihexylporphyrin (25 mg, 19 μmol) was dissolved in 5 ml methanol. Then a solution of sodium methanolate in dry methanol (1.0 ml, 0.02 N) was added. After 4 h, the solvent was evaporated under reduced pressure and the crude product was purified by flash chromatography, using dichloromethane/methanol 8:2 as the eluent. The desired product (17 mg, 91%) was obtained as a violet crystalline solid.

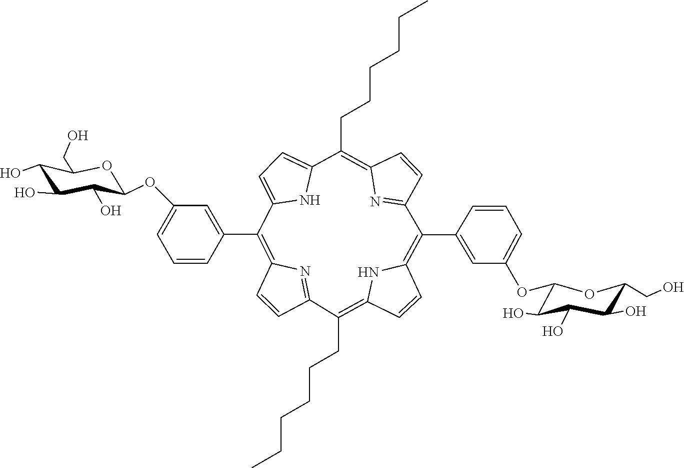

mp: >300° C., ¹H NMR (500 MHz, CD₃OD): δ=0.86-0.91 (br m, 6H, 2×CH₃), 1.31-1.44 (br m, 8H, 4×CH₂), 1.66-1.74 (br m, 4H, 2×CH₂), 2.33-2.43 (br m, 4H, 2×CH₂), 3.40-3.89 (br m, 10H, H‘ose’), 4.62-4.65 (br m, 2H, H‘ose’), 4.85-4.93 (4H, 2×CH₂), 5.22-5.25 (br m, 2H, H‘ose’), 7.57-7.61 (br m, 2H, Ar—H), 7.66-7.70 (br m, 2H, Ar—H), 7.75-7.80 (br m, 2H, Ar—H), 7.90-7.94 (br m, 2H, Ar—H), 8.80-8.92 (br m, 4H, β-H), 9.34-9.46 (br m, 4H, β-H) ppm. ESI-HRMS: C₅₆H₆₇N₄O₁₂⁺ ([M+H]⁺): calculated 987.4750, found 987.4723. UV/vis ((CH₃)₂CO): λₘₐₓ (log ε/dm³ mol⁻¹ cm⁻¹): 416 (5.18), 514 (3.86), 548 (3.58), 593 (3.30), 650 (3.40) nm.

### 2.4 Preparation of 5,15-bis-(4-β-D-glucosylphenyl)-10,20-dihexylporphyrin

In a typical experiment, under argon atmosphere, 5,15-bis-[4-(2,3,4,6-tetraacetyl-β-D-glucosyl)-phenyl]-10,20-dihexylporphyrin (50 mg, 38 μmol) was dissolved in 9.0 ml dry tetrahydrofuran and 9.0 ml methanol. Then a solution of sodium methanolate in dry methanol (3.0 ml, 0.02 N) was added. After 4 h, the solvent was evaporated under reduced pressure and the crude product was purified by flash chromatography, using dichloromethane/methanol 8:2 as the eluent. The desired product (36 mg, 97%) was obtained as a violet crystalline solid.

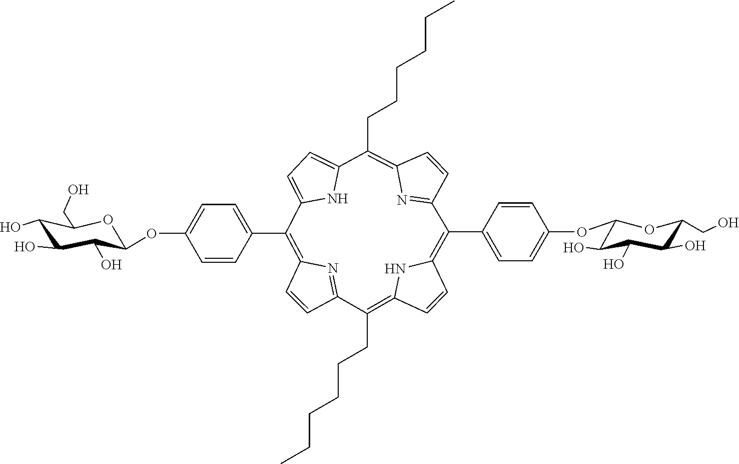

mp: 226° C., ¹H NMR (500 MHz, (CD₃)₂SO): δ=−2.92 (br s, 2H, NH), 0.84 (t, J=7.3 Hz, 6H, 2×CH₃), 1.24-1.30 (m, 4H, 4×CH₂), 1.35-1.41 (m, 4H, 4×CH₂), 1.69-1.75 (m, 4H, 4×CH₂), 2.32-2.37 (m, 4H, 4×CH₂), 3.38-3.43 (m, 4H, H‘ose’), 3.48-3.53 (m, 2H, H‘ose’), 3.55-3.61 (m, 2H, H‘ose’), 3.79-3.83 (m, 2H, H‘ose’), 4.69-4.72 (m, 2H, H‘ose’), 4.91-4.98 (m, 4H, 2×CH₂), 5.25 (d, J=7.4 Hz, 2H, H-1‘ose’), 7.46 (d, J=8.6 Hz, 4H, Ar—Hₘₑₜₐ), 8.08 (d, J=8.6 Hz, 4H, Ar—Hₒᵣₜₕₒ), 8.82 (d, J=4.8 Hz, 4H, β-H), 9.64 (d, J=4.8 Hz, 4H, β-H) ppm. ¹³C NMR (126 MHz, (CD₃)₂SO): δ=14.53 (q, CH₃), 22.72 (t, CH₂), 29.79 (t, CH₂), 29.94 (t, CH₂), 31.90 (t, CH₂), 39.24 (t, CH₂), 61.35 (t, C-6‘ose’), 70.36 (d, C‘ose’), 74.00 (d, C‘ose’), 77.26 (d, C‘ose’), 77.80 (d, C‘ose’), 101.12 (d, C-1‘ose’), 114.97 (d, Ar—Cₘₑₜₐ), 118.78 (s, Ar—Cₘₑₛₒ), 120.49 (s, Alkyl-Cₘₑₛₒ), 135.68 (d, Ar—Cₒᵣₜₕₒ), 157.90 (s, Ar—C_{OGlu}) ppm. ESI-HRMS: CO₅₆H₆₇N₄O₁₂⁺ ([M+H]⁺): calculated 987.4750, found 987.4746. UV/vis ((CH₃)₂SO): λₘₐₓ (log ε/dm³ mol⁻¹ cm⁻¹): 420 (5.19), 519 (3.89), 554 (3.73), 597 (3.43), 654 (3.56) nm.

### 2.5 Preparation of 5,15-bis-(4-β-D-galactosylphenyl)-10,20-dihexylporphyrin

In a typical experiment, under argon atmosphere, 5,15-bis-[4-(2,3,4,6-tetraacetyl-β-D-galactosyl)-phenyl]-10,20-dihexylporphyrin (30 mg, 23 μmol) was dissolved in 5.0 ml dry tetrahydrofuran and 5.0 ml methanol. Then a solution of sodium methanolate in dry methanol (1.8 ml, 0.02 N) was added. After 4 h, the solvent was evaporated under reduced pressure and the crude product was purified by flash chromatography, using dichloromethane/methanol 8:2 as the eluent. The desired product (21 mg, 93%) was obtained as a violet crystalline solid.

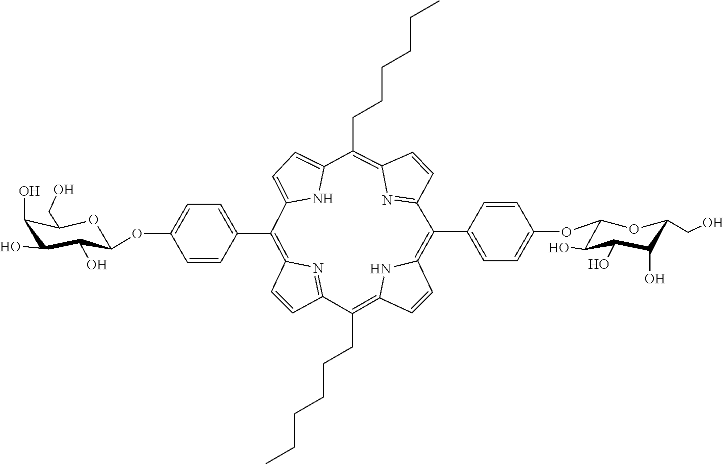

mp: 176° C., ¹H NMR (500 MHz, (CD₃)₂SO): δ=−2.89 (br s, 2H, NH), 0.86 (t, J=7.5 Hz, 6H, 2×CH₃), 1.28-1.35 (m, 4H, 2×CH₂), 1.39-1.44 (m, 4H, 2×CH₂), 1.71-1.78 (m, 4H, 2×CH₂), 2.35-2.42 (m, 4H, 2×CH₂), 3.56-3.58 (m, 2H, 2×H-5‘ose’), 3.67-3.71 (m, 4H, 2×H-6‘ose’), 3.77-3.84 (m, 6H, 2×H-2‘ose’, 2×H-3‘ose’, 2×H-4‘ose’), 4.64 (d, J=4.7 Hz, 2H, 2×OH), 4.75-4.78 (m, 2H, 2×OH), 4.92-4.98 (m, 4H, 2×CH₂), 4.99 (d, J=5.6 Hz, 2H, 2×OH), 5.20 (d, J=7.8 Hz, 2H, 2×H-1‘ose’), 5.40 (d, J=5.2 Hz, 2H, 2×OH), 7.48 (d, J=8.5 Hz, 4H, Ar—Hₘₑₜₐ), 8.09 (d, J=8.5 Hz, 4H, Ar—Hₒᵣₜₕₒ), 8.84 (d, J=4.4 Hz, 4H, β-H), 9.65 (d, J=4.4 Hz, 4H, β-H) ppm. ¹³C NMR (126 MHz, (CD₃)₂SO): δ=14.52 (q, CH₃), 22.71 (t, CH₂), 29.94 (t, CH₂), 30.98 (t, CH₂), 31.89 (t, CH₂), 34.91 (t, CH₂), 39.22 (t, CH₂), 61.12 (t, C-6‘ose’), 68.86 (d, C‘ose’), 71.08 (d, C‘ose’), 74.01 (d, C-5‘ose’), 76.31 (d, C‘ose’), 101.83 (d, C-1‘ose’), 115.03 (d, Ar—Cₘₑₜₐ), 118.81, 120.47, 135.67 (d, Ar—Cₒᵣₜₕₒ), 158.02 (s, Ar—C_{OGal}) ppm. ESI-HRMS: CO₅₆H₆₇N₄O₁₂⁺ ([M+H]⁺): calculated 987.4750, found 987.4697. UV/vis ((CH₃)₂SO): λₘₐₓ (log ε/dm³ mol⁻¹ cm⁻¹): 421 (5.19), 519 (3.92), 555 (3.76), 597 (3.47), 654 (3.56) nm.

### 2.6 Preparation of 5,15-bis-(3-β-D-glucosylphenyl)-10,20-diphenylporphyrin

In a typical experiment, under argon atmosphere, 5,15-bis-[3-(2,3,4,6-tetraacetyl-β-D-glucosyl)-phenyl]-10,20-diphenylporphyrin (20 mg, 15 μmol) was dissolved in 4.0 ml dry tetrahydrofuran and 4.0 ml methanol. Then a solution of sodium methanolate in dry methanol (1.6 ml, 0.02 N) was added. After 4 h, the solvent was evaporated under reduced pressure and the crude product was purified by flash chromatography, using dichloromethane/methanol 8:2 as the eluent. The desired product (13 mg, 89%) was obtained as a violet crystalline solid.

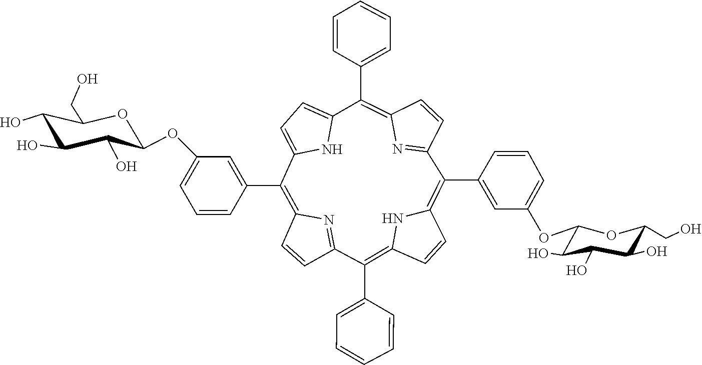

mp: >300° C., ¹H NMR (700 MHz, (CD₃)₂SO): δ=−2.92 (br s, 2H, NH), 3.20-3.24 (m, 2H, H‘ose’), 3.29-3.35 (m, 6H, H‘ose’), 3.46-3.50 (m, 2H, H-6_{A}‘ose’), 3.66-3.69 (m, 2H, H-6_{B}‘ose’), 4.54-4.56 (m, 2H, OH‘ose’), 4.98-5.00 (m, 2H, OH‘ose’), 5.09 (d, J=4.9 Hz, 1H, OH‘ose’), 5.19-5.21 (m, 2H, H-1‘ose’), 5.42 (d, J=4.8 Hz, 1H, OH‘ose’), 7.52-7.55 (m, 2H, Ar—H), 7.72-7.75 (m, 2H, Ar—H), 7.83-7.89 (m, 8H, 4×Ph-Hₘₑta, 2×Ph-Hₚₐᵣₐ, 2×Ar—H), 7.89-7.91 (m, 2H, Ar—H), 8.23-8.25 (m, 4H, Ph-Hₒᵣₜₕₒ), 8.84-8.93 (m, 8H, β-H) ppm. ¹³C NMR (176 MHz, (CD₃)₂SO): δ=61.11 (t, C-6‘ose’), 70.16 (d, C‘ose’), 73.87 (d, C‘ose’), 77.03 (d, C‘ose’), 77.44 (d, C‘ose’), 100.83-100.85 (d, C-1‘ose’), 116.28 (d, Ar—C), 120.06 (s, Ar—Cₘₑₛₒ), 120.46 (s, Ph-Cₘₑₛₒ), 122.88 (d, Ar—C), 127.50 (d, Ph-Cₘₑₜₐ), 128.34 (d, Ar—C), 128.58 (d, Ph-Cₚₐᵣₐ), 128.93 (d, Ar—C), 134.66-134.72 (d, Ph-Cₒᵣₜₕₒ), 141.66 (s, Ph-Cᵢₚₛₒ), 142.82 (s, Ar—Cᵢₚₛₒ), 156.35 (s, Ar—C_{OGlu}) ppm. ESI-HRMS: CO₅₆H₅₁N₄O₁₂⁺ ([M+H]⁺): calculated 971.3503, found 971.3530. UV/vis ((CH₃)₂SO): λₘₐₓ (log ε/dm³ mol⁻¹ cm⁻¹): 416 (5.26), 513 (3.84), 547 (3.54), 590 (3.32), 649 (3.30) nm.

### 2.7 Preparation of 5,10,15,20-tetrakis-(4-β-D-glucosyl)-porphyrin (Reference Example)

In a typical experiment, under argon atmosphere, 5,10,15,20-tetrakis-[4-(2,3,4,6-tetraacetyl-β-D-glucosyl)phenyl]-porphyrin (30 mg, 15 μmol) was dissolved in 6 ml dry tetrahydrofuran and 6 ml methanol. Then, a solution of sodium methanolate in dry methanol (0.6 ml, 0.12 N) was added. After 4 h, the solvent was evaporated under reduced pressure and the crude product was purified by RP₁₈ flash chromatography, using methanol/water 9:1 as the eluent. The desired product (17 mg, 84%) was obtained as a violet crystalline solid.

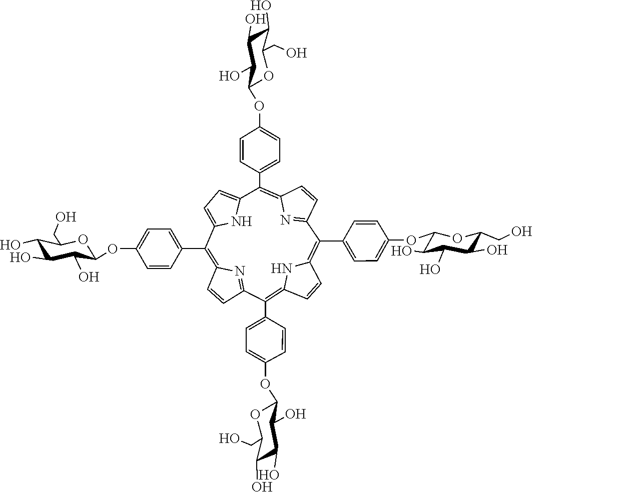

mp: >300° C., ¹H NMR (500 MHz, (CD₃)₂SO): δ=−2.91 (br s, 2H, NH), 3.27-3.31 (m, 4H, H‘ose’), 3.40-3.45 (m, 8H, H‘ose’), 3.49-3.54 (ddd, J=1.9, 5.7, 9.5 Hz, 4H, H-5‘ose’), 3.56-3.62 (dd, J=5.7, 11.8 Hz, 4H, H-6_{B}‘ose’), 3.79-3.83 (m, 4H, H-6_{A}‘ose’), 4.73 (t, J=5.8 Hz, 4H, OH‘ose’), 5.12 (d, J=5.4 Hz, 4H, OH‘ose’), 5.21 (d, J=4.2 Hz, 4H, OH‘ose’), 5.23 (d, J=7.2 Hz, 4H, H-1 ‘ose’), 5.52 (d, J=4.7 Hz, 4H, OH‘ose’), 7.48 (d, J=8.5 Hz, 8H, 8×Ar-Hₘₑₜₐ), 8.13 (d, J=8.5 Hz, 8H, 8×Ar-Hₒᵣₜₕₒ), 9.87 (s, 8H, β-H) ppm. ¹³C NMR (126 MHz, (CD₃)₂SO): δ=61.36 (t, C-6‘ose’), 70.35 (d, C‘ose’), 74.01 (d, C‘ose’), 77.26 (d, C‘ose’), 77.79 (d, C‘ose’), 101.13 (d, C-1‘ose’), 115.11 (d, Ar—Cₘₑₜₐ), 120.18 (s, Ar—Cₘₑₛₒ), 135.21 (s, Ar—Cᵢₚₛₒ), 135.78 (d, Ar—Cₒᵣₜₕₒ), 158.06 (s, Ar—C_{OGlu}) ppm. ESI-HRMS: C₆₈H₇₁N₄O₂₄⁺ [M+H]⁺: calculated 1327.4458, found 1327.4490. UV/vis ((CH₃)₂SO): λₘₐₓ (log ε/dm³ mol⁻¹ cm⁻¹): 423 (5.67), 518 (4.62), 555 (4.51), 594 (4.33), 650 (4.31) nm.

### 2.8 Preparation of 5,10,15,20-tetrakis-(3-β-D-galactosyl)-porphyrin (Reference Example)

In a typical experiment, under argon atmosphere, 5,10,15,20-tetrakis-[3-(2,3,4,6-tetraacetyl-β-D-galactosyl)phenyl]-porphyrin (55 mg, 28 μmol) was dissolved in 2 ml dry tetrahydrofuran and 10 ml methanol. Then a solution of sodium methanolate in dry methanol (1.0 ml, 0.12 N) was added. After 4 h, the solvent was evaporated under reduced pressure and the crude product was purified by RP₁₈ flash chromatography, using methanol/water 9:1 as the eluent. The desired product (30 mg, 82%) was obtained as a violet crystalline solid.

This porphyrin is an atropisomer.

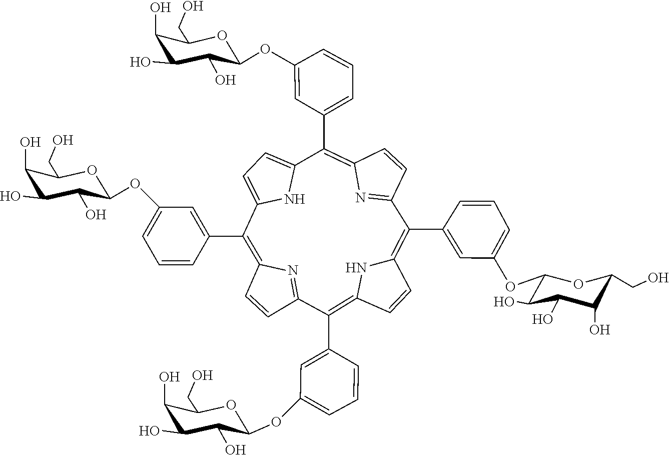

mp: 253° C., ¹H NMR (700 MHz, (CH₃)₂SO): δ=−2.95 (br s, 2H, NH), 3.42-3.45 (m, 4H, H‘ose’), 3.48-3.52 (m, 4H, H‘ose’), 3.55-3.59 (m, 8H, H‘ose’), 3.66-3.70 (m, 8H, H‘ose’), 4.59 (br s, 8H, OH-‘ose’), 4.92 (br s, 4H, OH-‘ose’), 5.12-5.18 (m, 4H, H-1‘ose’), 5.30 (br s, 4H, OH-‘ose’), 7.50-7.53 (m, 4H, Ar), 7.71-7.75 (m, 4H, Ar), 7.79-7.84 (m, 4H, Ar), 7.87-7.90 (m, 4H, Ar), 8.87-8.93 (m, 8H, β-H) ppm. ESI-HRMS: C₆₈H₇₀N₄O₂₄Na⁺ ([M+Na]⁺): calculated 1349.4278, found 1349.4297. UV/vis ((CH₃)₂CO): λₘₐₓ (log ε/dm³ mol⁻¹ cm⁻¹): 416 (5.09), 513 (3.67), 546 (3.45), 589 (3.36), 644 (3.30) nm.

### 2.9 Preparation of 5-(3-β-D-glucosylphenyl)-10,15,20-tris-(4-1′-thio-β-D-glucosyl-2,3,5,6-tetra-fluorophenyl)-porphyrin

In a typical experiment, under argon atmosphere, 5-(4-β-D-glucosylphenyl)-10,15,20-tris-(pentafluorophenyl)-porphyrin (30 mg, 28 μmol) and 1-thio-β-D-glucose sodium salt (20 mg, 93 μmol) were reacted in 4 ml dry DMF overnight at room temperature. Purification was achieved by column chromatography on silica using ethyl acetate/methanol (17:3) as the eluent. The analytically pure product (40 mg, 89%) was obtained as a violet crystalline solid.

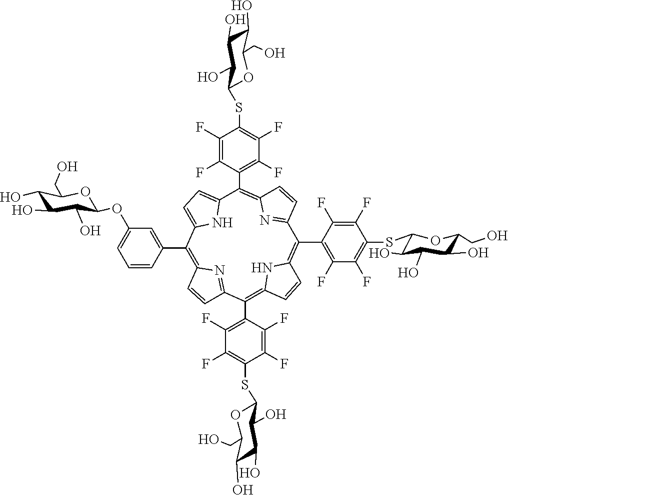

mp: 243° C., ¹H NMR (700 MHz, CD₃OD): δ=−2.89 (s, 2H, NH), 3.45-3.63 (m, 16H, H-2′‘ose’, H-2‘ose’, H-3′‘ose’, H-3‘ose’, H-4′‘ose’, H-4‘ose’, H-5′‘ose’, H-5‘ose’), 3.72 (dd, J=4.8, 11.9 Hz, 1H, H-6_{B}‘ose’), 3.82 (dd, J=6.2, 12.0 Hz, 2H, H-6_{B}‘ose’), 3.83 (dd, J=6.2, 12.0 Hz, 1H, H-6_{B}‘ose’), 3.88 (dd, J=1.9, 12.0 Hz, 1H, H-6_{A}‘ose’), 4.05 (dd, J=2.2, 11.9 Hz, 2H, H-6_{A}′‘ose’), 4.06 (dd, J=2.2, 11.9 Hz, 1H, H-6_{A}′‘ose’), 5.19-5.23 (m, 3H, H-1′‘ose’), 5.27 (d, J=7.7 Hz, 1H, H-1‘ose’), 7.59-7.63 (m, 1H, Ar—H), 7.64-7.68 (m, 1H, Ar—H), 7.81-7.84 (m, 1H, Ar—H), 8.02-8.05 (m, 1H, Ar—H), 8.98-9.30 (m, 8H, β-H) ppm. ¹³C NMR (176 MHz, (CD₃OD): 6=61.04 (C-6‘ose’), 61.70 (C-6′‘ose’), 69.96 (C‘ose’), 70.32 (C-4′‘ose’), 73.63 (C‘ose’), 74.59 (C-2′‘ose’), 76.56 (C‘ose’), 76.68 (C‘ose’), 78.37 (C-3′‘ose’), 81.37 (C-5′‘ose’), 85.41 (C-1′‘‘ose’), 100.75 (C-1‘ose’), 102.86 (Ar_{F}—Cₘₑₛₒ), 103.92 (Ar_{F}—Cₘₑₛₒ), 113.41-113.64 (Ar_{F}—C_{SGlu}), 116.31 (Ar—C), 120.53-120.93 (Ar_{F}—Cᵢₚₛₒ), 122.41 (Ar—Cₘₑₛₒ), 122.83 (Ar—C), 127.59 (Ar—C), 128.78 (Ar—C), 142.15 (Ar—Cᵢₚₛₒ), 145.58-145.67 (Ar_{F}—C), 146.43-146.56 (Ar_{F}—C), 146.98-147.07 (Ar_{F}—C), 147.87-147.95 (Ar_{F}—C), 156.20 (Ar—C_{OGlu}) ppm. ¹⁹F NMR (471 MHz, CD₃OD): δ=−140.47-−140.28 (m, 6 F, Ar—Fₘₑₜₐ), −135.11-−134.98 (m, 6 F, Ar—Fₒᵣₜₕₒ) ppm. ESI-HRMS: C₆₈H₅₈F₁₂N₄NaO₂₁S₃⁺ ([M+Na]⁺): calculated 1613.2462, found 1613.2351. UV/vis (CH₃OH): λₘₐₓ(log ε/dm³ mol⁻¹ cm⁻¹): 411 (5.53), 506 (4.42), 580 (3.77), 636 (3.11) nm.

### 2.10 Preparation of 5-(3-β-D-glucosylphenyl)-10,15,20-tris-(4-1′-thio-β-D-galactosyl-2,3,5,6-tetra-fluorophenyl)-porphyrin

In a typical experiment, under argon atmosphere, 5-(4-β-D-glucosylphenyl)-10,15,20-tris-(pentafluorophenyl)-porphyrin (30 mg, 28 μmol) and 1-thio-β-D-galactose sodium salt (20 mg, 93 μmol) were reacted in 4 ml dry DMF overnight at room temperature. Purification was achieved by column chromatography on silica using methanol/water (8:2) as the eluent. The analytically pure product (36 mg, 81%) was obtained as a violet crystalline solid.

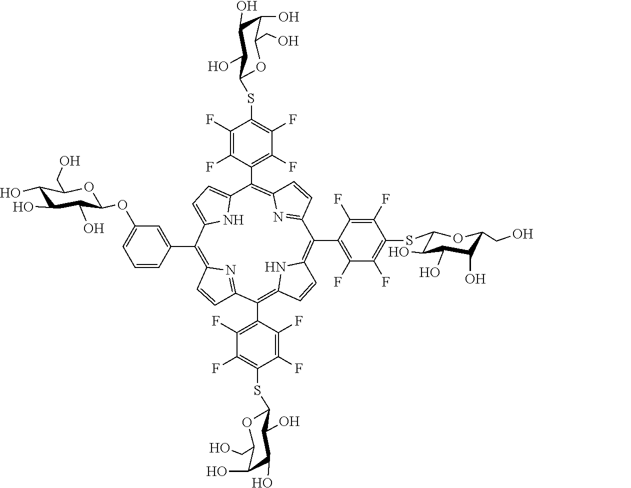

mp: 267° C. ¹H NMR (700 MHz, CD₃OD): δ=−2.92 (s, 2H, NH), 3.44-3.47 (m, 1H, H-5‘ose’), 3.48 (dd, J=8.4, 9.8 Hz, 1H, H-4‘ose’), 3.54 (dd, J=8.4, 9.0 Hz, 1H, H-3‘ose’), 3.61 (dd, J=7.8, 9.0 Hz, 1H, H-2‘ose’), 3.69 (dd, J=3.3, 9.3 Hz, 3H, H-3′‘ose’), 3.72 (dd, J=4.9, 12.0 Hz, 1H, H-6_{B}‘ose’), 3.75-3.78 (m, 3H, H-5′‘ose’), 3.85-3.93 (m, 10H, 1×H-6_{A}‘ose’, 3×H-2′‘ose’, 3×H-6_{A}′‘ose’, 3×H-6B′‘ose’), 4.03-4.05 (m, 3H, H-4′‘ose’), 5.12 (d, J=9.5 Hz, 3H, H-1′‘ose’), 5.26 (d, J=7.8 Hz, 1H, H-1‘ose’), 7.58-7.62 (m, 1H, Ar—H), 7.64-7.68 (m, 1H, Ar—H), 7.78-7.81 (m, 1H, Ar—H), 8.00-8.03 (m, 1H, Ar—H), 8.93-9.32 (m, 8H, β-H) ppm. ¹³C NMR (176 MHz, (CD₃OD): δ=61.00 (C-6‘ose’), 61.33 (C-6′‘ose’), 69.22 (C-4′‘ose’), 69.93 (C-4‘ose’), 71.43 (C-2′‘ose’), 73.60 (C-2‘ose’), 74.94 (C-3′‘ose’), 76.52 (C-3‘ose’), 76.63 (C-5‘ose’), 79.91 (C-5′‘ose’), 86.28 (C-1′‘ose’), 100.70 (C-1‘ose’), 102.86 (Ar_{F}—Cₘₑₛₒ), 103.92 (Ar_{F}—Cₘₑₛₒ), 113.28-113.52 (Ar_{F}—C_{SGal}), 116.31 (Ar—C), 120.65-121.05 (Ar_{F}—Cᵢₚₛₒ), 122.39 (Ar—Cₘₑₛₒ), 122.82 (Ar—C), 127.62 (Ar—C), 128.80 (Ar—C), 142.11 (Ar—Cᵢₚₛₒ), 145.56-145.66 (Ar_{F}—C), 146.63-146.71 (Ar_{F}—C), 146.96-147.05 (Ar_{F}—C), 148.01-148.10 (Ar_{F}—C), 156.14 (Ar—C_{OGlu}) ppm. ¹⁹F NMR (471 MHz, CD₃OD): δ=−140.52-−140.32 (m, 6 F, Ar—Fₘₑₜₐ), −134.34-−134.70 (m, 6 F, Ar—Fₒᵣₜₕₒ) ppm. ESI-HRMS: C₆₈H₅₈F₁₂N₄NaO₂₁S₃⁺ ([M+Na]⁺): calculated 1613.2462, found 1613.2384. UV/vis (CH₃OH): λₘₐₓ (log ε/dm³ mol⁻¹ cm⁻¹): 412 (5.57), 506 (4.29), 581 (3.81), 636 (2.98) nm.

### Example 3—Preparation of Glycosylated Chlorins

### 3.1 Preparation of 5-(3-β-D-glucosylphenyl)-10,15,20-trihexyl-17,18-dihydroxy-17,18-chlorin

In a typical experiment, osmium tetroxide (37 mg, 0.2 mmol) was added to a stirred solution of 5-[3-(2,3,4,6-tetraacetyl-β-D-glucosyl)phenyl]-10,15,20-trihexylporphyrin (120 mg, 0.12 mmol) in dichloromethane/pyridine 2:1 (6 ml). After stirring for 30 minutes at 0° C. and additional 8 hours at room temperature, a saturated solution of sodium bisulfite in water/methanol 1:1 (25 ml) was added and the mixture was stirred for 18 h. The reaction mixture was filtered through Celite and dried over anhydrous sodium sulfate. The solvent was evaporated and the residue was purified by flash chromatography with dichloromethane/ethyl acetate 95:5 as eluent, followed by recrystallization from dichloromethane/methanol. The chlorin (30 mg, 24%) was obtained as a violet crystalline solid after recrystallization from dichloromethane/aqueous methanol, as a regioisomeric mixture.

To a stirred solution of 5-[3-(2,3,4,6-tetraacetyl-β-D-glucosyl)phenyl]-10,15,20-trihexyl-17,18-dihydroxy-17,18-chlorin (25 mg, mmol) in dry tetrahydrofuran/methanol 1:1 (5 ml) under an argon atmosphere, a solution of sodium methanolate in dry methanol (1.0 ml, 0.02 N) was added. After 3 h, the solvent was evaporated under reduced pressure and the crude product was purified by RP₁₈ flash chromatography, using methanol/water 9:1 as the eluent. The desired product (19 mg, 91%) was obtained after recrystallization from dichloromethane/aqueous methanol as a violet crystalline solid.

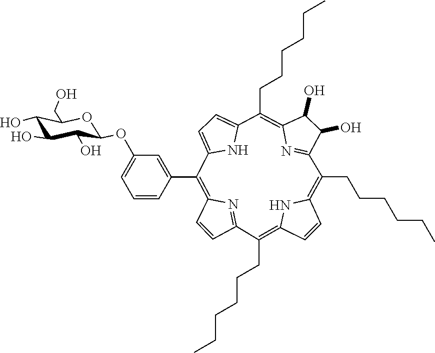

mp: 135° C., ¹H NMR (500 MHz, (CD₃)₂CO): δ=−1.95 (s, 1H, NH), −1.91 (s, 1H, NH), 0.88-0.98 (m, 9H, 3×CH₃), 1.32-1.55 (m, 12H, 6×CH₂), 1.73-1.85 (m, 6H, 3×CH₂), 2.21-2.44 (m, 6H, 3×CH₂), 3.52-3.63 (m, 5H), 3.67-3.74 (m, 1H), 3.82-3.88 (m, 1H), 4.18-4.20 (br s, 1H), 4.30-4.32 (m, 1H), 4.39-4.49 (m, 2H, CH₂), 4.61-4.67 (m, 2H, CH₂), 4.68-4.70 (m, 1H), 4.74-4.80 (m, 2H, CH₂), 5.25-5.28 (m, 3H), 6.57-6.60 (m, 2H, β-H), 7.47-7.51 (m, 1H, Ar—H), 7.63-7.67 (m, 1H, Ar—H), 7.71-7.73 (m, 1H, Ar—H), 7.79-7.82 (m, 1H, Ar—H), 8.47-8.49 (m, 1H, β-H), 8.71-8.73 (m, 1H, β-H), 9.15-9.18 (m, 2H, β-H), 9.24-9.26 (m, 1H, β-H), 9.49-9.51 (m, 1H, β-H) ppm. ESI-HRMS: C₅₀H₆₇N₄O₈⁺ [M+H]⁺: calculated 851.5, found 851.5. UV/vis ((CH₃)₂CO): λₘₐₓ(log ε/dm³ mol⁻¹ cm⁻¹): 406 (5.32), 428 (5.21), 525 (4.25), 551 (4.34), 596 (3.95), 649 (4.37) nm.

### 3.2 Preparation of 5,10,15-tris-(3-β-D-galactosylphenyl)-20-[3,5-bis-(trifluoromethyl)-phenyl]-17,18-dihydroxy-17,18-chlorin

In a typical experiment, osmium tetroxide (100 mg, 0.39 mmol) was added to a stirred solution of 5,10,15-tris-[3-(2,3,4,6-tetraacetyl-β-D-galactosyl)phenyl]-20-[3,5-bis-(trifluoromethyl)phenyl]-porphyrin (300 mg, 0.17 mmol) in dichloromethane/pyridine 1:1 (26 ml). After stirring for 30 minutes at 0° C. and additional 2 hours at room temperature, a saturated solution of sodium bisulfite in water/methanol 1:1 (25 ml) was added and the mixture was stirred for 18 h. The reaction mixture was filtered through Celite and dried over anhydrous sodium sulfate. The solvent was evaporated and the residue was purified by flash chromatography with dichloromethane/methanol 95:5 as eluent, followed by recrystallization from dichloromethane/aqueous methanol. The chlorin (129 mg, 42%) was obtained as a violet crystalline solid after recrystallization from dichloromethane/aqueous methanol, as a regioisomeric mixture.

To a stirred solution of 5,10,15-tris-[3-(2,3,4,6-tetraacetyl-β-D-galactosyl)phenyl]-20-[3,5-bis-(trifluoromethyl)phenyl]-17,18-dihydroxy-17,18-chlorin (46 mg, 25 μmol) in dry tetrahydrofuran/methanol 1:1 (10 ml) under an argon atmosphere, a solution of sodium methanolate in dry methanol (1.0 ml, 0.1 N) was added. After 4 h, the solvent was evaporated under reduced pressure and the crude product was purified by RP₁₈ flash chromatography, using methanol/water 85:15 as the eluent. The desired product (33 mg, 99%) was obtained as a violet crystalline solid after washing with dichloromethane.

This chlorin is an atropisomer.

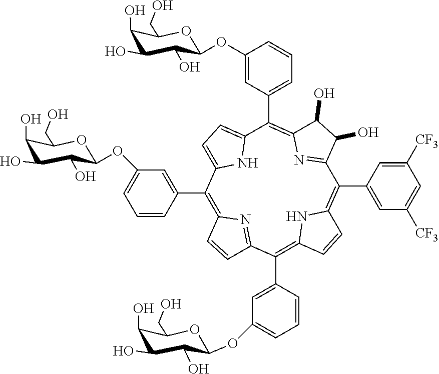

mp: >300° C., ¹H NMR (500 MHz, CD₃OD): δ=3.54-3.88 (m, 18H, H‘ose’), 5.02-5.12 (3H, H‘ose’), 6.13-6.36 (m, 2H, β-H), 7.39-8.79 (m, 21H, 6×β-H, 15 Ar—H) ppm. ESI-HRMS: C₆₄H₆₀F₆N₄O₂₀Na⁺ ([M+Na]⁺): calculated 1341.3597, found 1341.3594. UV/vis ((CH₃)₂CO): λₘₐₓ (log ε/dm³ mol⁻¹ cm⁻¹): 407 (4.51), 515 (3.53), 541 (3.49), 594 (3.18), 646 (3.78) nm.

### 3.3 Preparation of 5,10,15-tris-(3-β-D-lactosylphenyl)-20-[3,5-bis-(trifluoromethyl)-phenyl]17,18-dihydroxy-17,18-chlorin

In a typical experiment, osmium tetroxide (100 mg, 0.39 mmol) was added to a stirred solution of 5,10,15-tris-[3-(2,3,4,6,2′,3′,6′-heptaacetyl-β-D-lactosyl)-phenyl]-20-[3,5-bis-(trifluoro-methyl)phenyl]-porphyrin (350 mg, 0.13 mmol) in dichloromethane/pyridine 1:1 (15 ml). After stirring for 30 minutes at 0° C. and additional 2 hours at room temperature, a saturated solution of sodium bisulfite in water/methanol 1:1 (25 ml) was added and the mixture was stirred for 18 h. The reaction mixture was filtered through Celite and dried over anhydrous sodium sulfate. The solvent was evaporated and the residue was purified by flash chromatography with dichloromethane/methanol 95:5 as eluent, followed by recrystallization from dichloromethane/aqueous methanol. The chlorin (39 mg, 8%) was obtained as a violet crystalline solid after recrystallization from dichloromethane/aqueous methanol, as a regioisomeric mixture.

To a stirred solution of 5,10,15-tris-[3-(2,3,4,6,2′,3′,6′-heptaacetyl-D-lactosyl)phenyl]-20-[3,5-bis-(trifluoromethyl)phenyl]-17,18-dihydroxy-17,18-chlorin (32 mg, 12 μmol) in dry tetrahydrofuran/methanol 1:1 (10 ml) under an argon atmosphere, a solution of sodium methanolate in dry methanol (1.5 ml, 0.1 N) was added. After 4 h, the solvent was evaporated under reduced pressure and the crude product was purified by RP₁₈ flash chromatography, using methanol/water 85:15 as the eluent. The desired product (21 mg, 98%) was obtained as a violet crystalline solid after washing with dichloromethane.

This chlorin is an atropisomer.

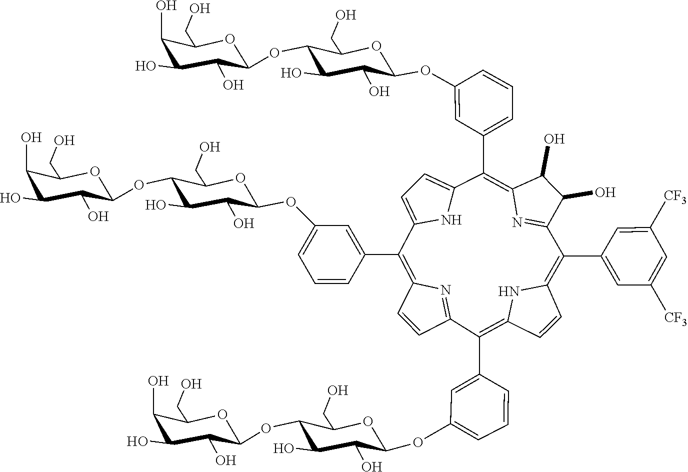

mp: >300° C., ¹H NMR (500 MHz, CD₃OD): δ=3.43-3.84 (m, 36H, H‘ose’), 4.31-4.38 (m, 3H, H‘ose’), 5.17-5.25 (m, 3H, H‘ose’), 6.13-6.32 (m, 2H, β-H), 7.39-8.80 (m, 21H, 6×β-H, 1 Ar—H) ppm. ESI-HRMS: C₈₂H₉₀F₆N₄O₃₅Na⁺ ([M+Na]⁺): calculated 1827.5182, found 1827.5282. UV/vis (CH₃CH₂OH): λₘₐₓ (log ε/dm³ mol⁻¹ cm⁻¹): 415 (4.92), 514 (3.86), 541 (3.83), 594 (3.49), 646 (4.12) nm.

### Example 4—Cell Tests of Selected Compounds in the HT 29 and Other Cell Lines

The photosensitizing activity was determined in the following cell lines:
HT29 (human colon adenocarcinoma cell line)
L929 (mouse fibroblast cell line)
A431 (human epidermoid carcinoma cell line)
A253 (submaxillary salivary gland, epidermoid cell line)
CAL-27 (human tongue squamous cell carcinoma cell line).

The cell lines were grown in DMEM (PAA Laboratories GmbH) supplemented with 10% heat-inactivated fetal calf serum (FCS, PAA Laboratories GmbH), 1% penicillin (10000 IU) and streptomycin (10000 μg/ml, PAA Laboratories GmbH). Cells were kept as a monolayer culture in a humidified incubator (5% CO₂ in air at 37° C.).

A photosensitizer stock solution (2 mM) was performed in DMSO and was kept in the dark at 4° C. Further dilution was performed in DMEM medium without phenol red supplemented with 10% FCS to reach a final photosensitizer concentration of 2 or 10 μM, respectively.

2·10⁴ cells/ml were seeded in micro plates (2·10⁵ cells/well). Cells were incubated with fresh medium (DMEM without phenol red) containing 10% FCS with 2 or 10 μM of the photosensitizer for 24 h before light exposure. Before photosensitization, cells were washed, cell culture medium was exchanged with DMEM without phenol red and 10% FCS, then irradiated at room temperature with a 652 nm diode laser (Ceralas PDT 652, biolitec AG) at a fixed fluence rate of 100 mW/cm² (50 J/cm²). Following irradiation, cells were incubated in a humidified incubator (5% CO₂ in air at 37° C.) for 24 h until cell viability assay.

The cell viability was assessed by the XTT assay. 500 mg XTT (sodium 3′-[phenylaminocarbonyl)-3,4-tetrazolium]-bis(4-methoxy-6-nitro)benzene sulfonic acid, Applichem GmbH) is dissolved in 500 ml PBS-Buffer (without Ca²⁺ and Mg²⁺) and sterile filtered. Solution was stored in the dark at −20° C. until use. A sterile solution containing PMS (N-methyl dibenzopyrazine methyl sulfate, Applichem GmbH) was needed as an activation reagent for the XTT. 0.383 mg PMS was dissolved in 1 ml PBS-Buffer. The solution should be stored frozen and should not be exposed to light. The XTT reagent solution was thawed in a 37° C. water bath and the activation solution (PMS) was added immediately prior to use. To prepare a reaction solution sufficient for one micro plate (96 wells), 0.1 ml activation solution (PMS) was given to 5 ml XTT reagent. The medium in the micro plate was exchanged with RPMI without phenol red and 10% FCS (100 μl) prior adding 50 μl XTT reaction solution per well. The micro plate was incubated for 2-3 hours at 37° C. and 5% CO₂ until an orange dye is to be formed. The micro plate has been shaken gently to evenly distribute the dye in the wells.

The absorbance of the samples was measured with a spectrophotometer (Infinite 200, Tecan Group Ltd.) at a wavelength of 490 nm. In order to measure reference absorbance (to measure non-specific readings) a wavelength of 630-690 nm was used.

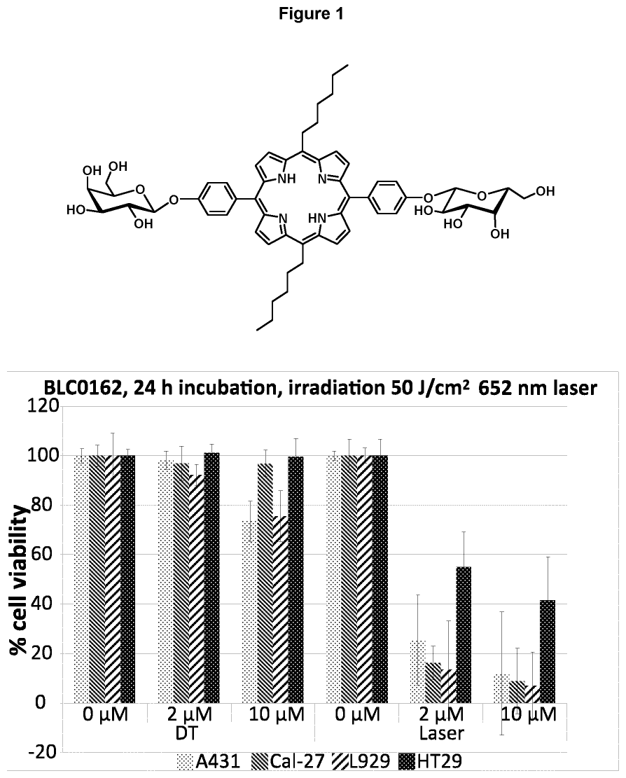

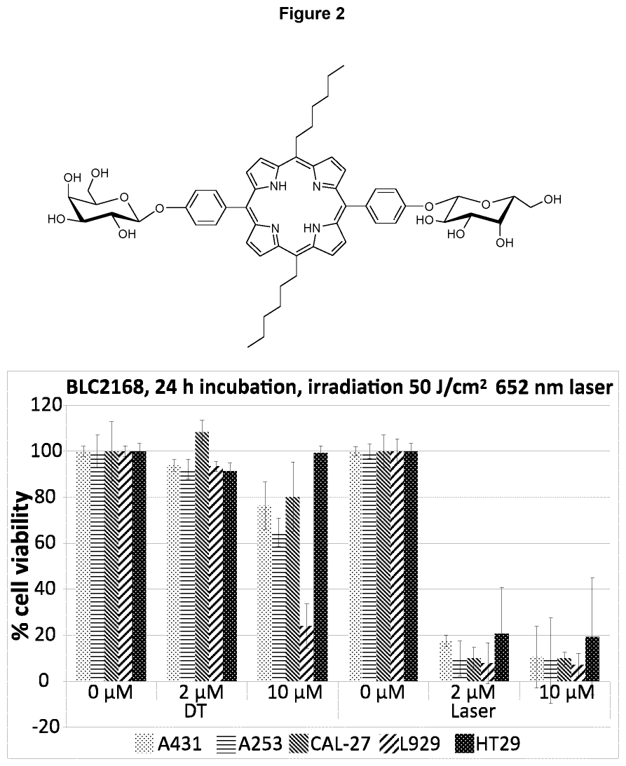

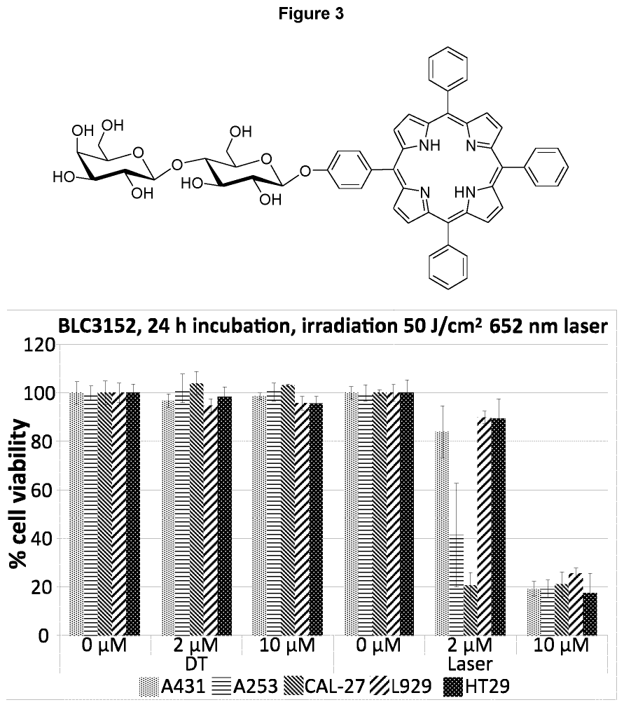

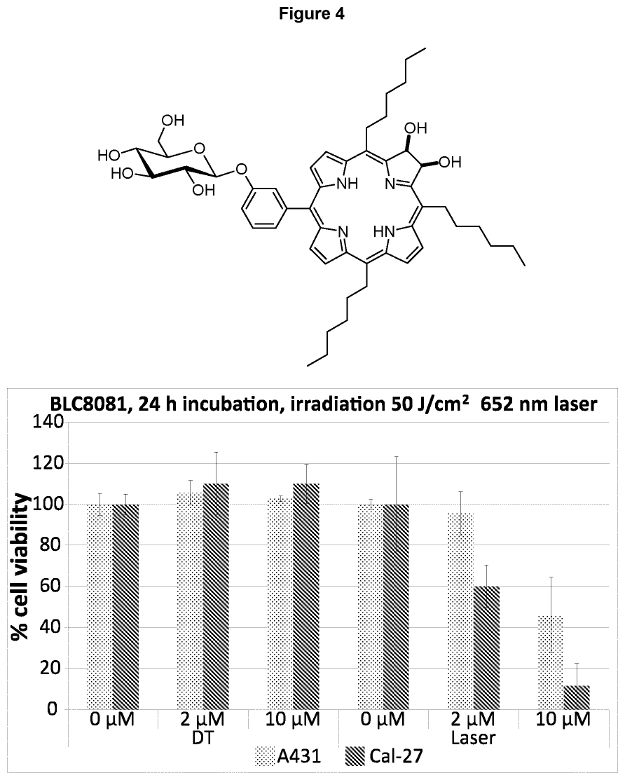

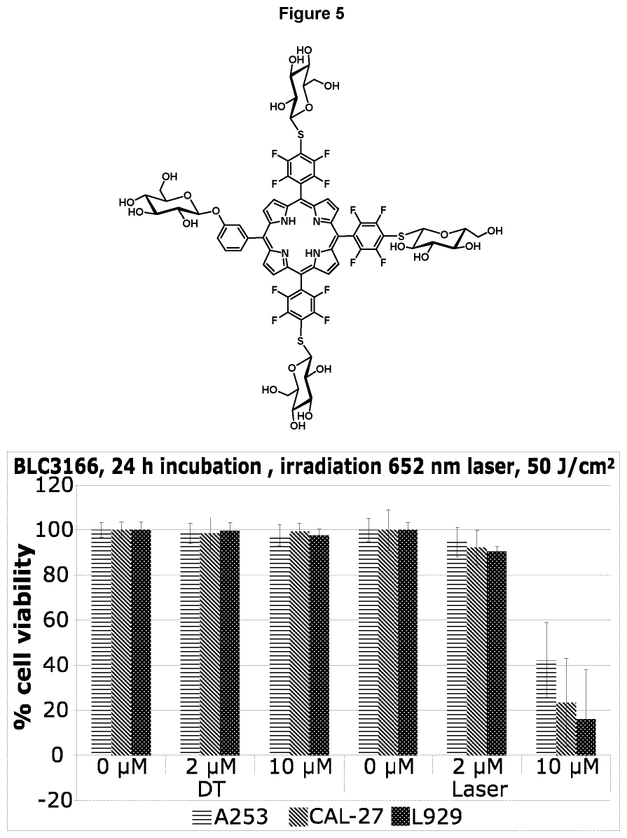

The results of Examples 4.1 to 4.5, which are shown in FIGS. 1 to 5, illustrate the photodynamic activity (“DT” means dark toxicity and “Laser” means photo toxicity) of photosensitizers having a substitution pattern according to the present invention. Specifically the photosensitizers according to the invention exhibit a strong photodynamic activity even in the HT29 cell line, which is known to be very resistant against cell-toxic agents and PDT as well.

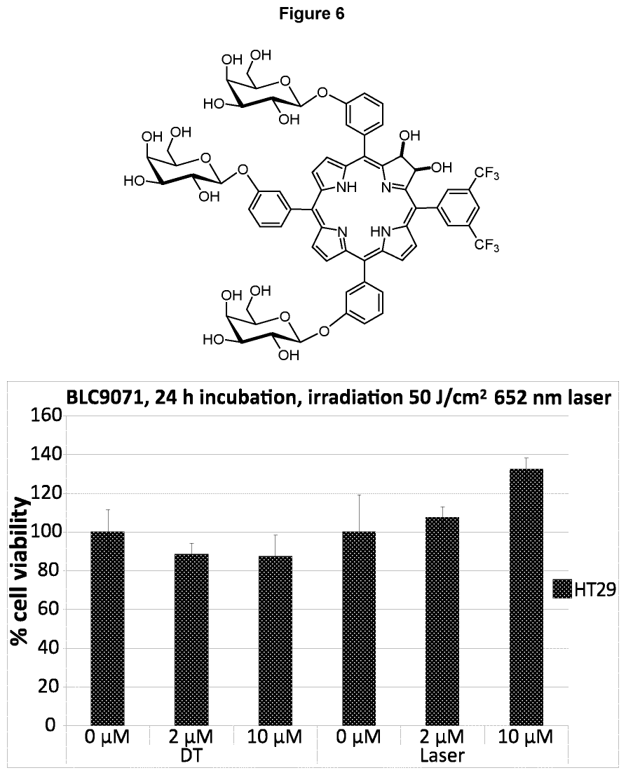

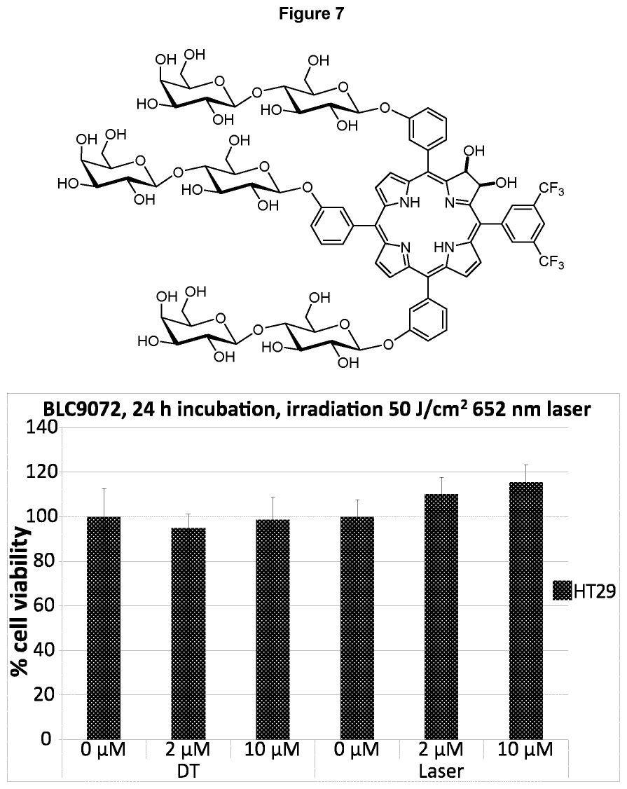

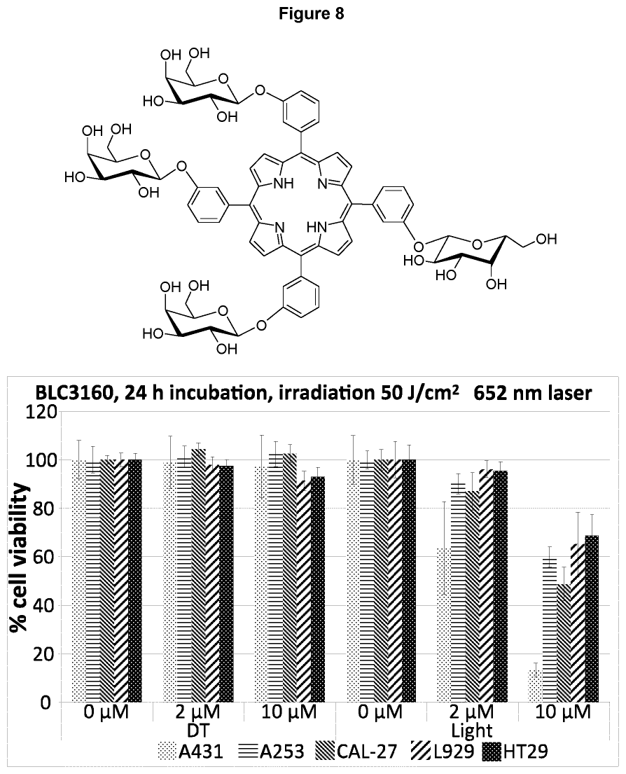

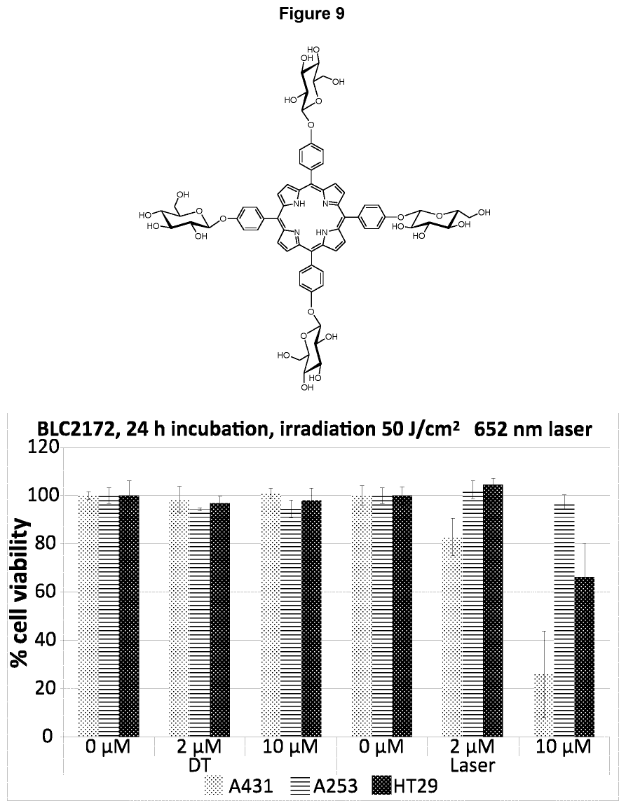

The results of Examples 4.6 to 4.9, which are shown in FIGS. 6 to 9, are included to illustrate, that photosensitizers which do not have a substitution pattern as referred to in the present invention exhibit a less promising photodynamic activity in the cell experiments, in particular with respect to cell line HT29.

1. A tetrapyrrolic compound having a structure of Formula 1, 2, or 3: 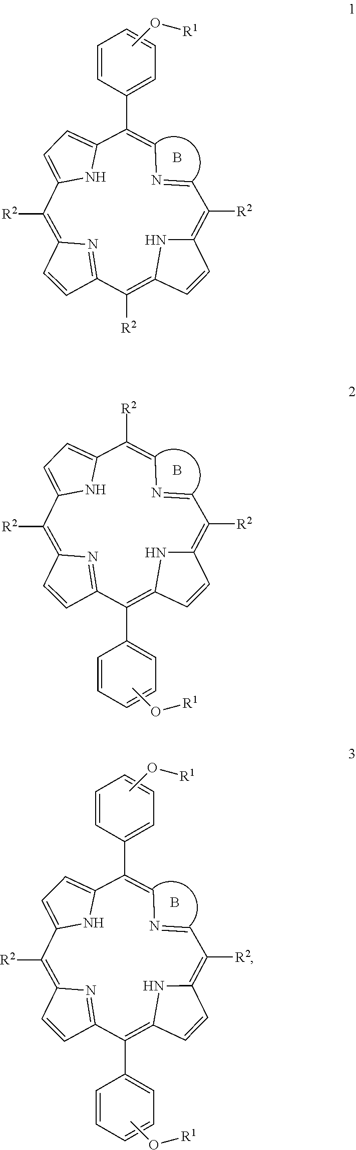
wherein B is 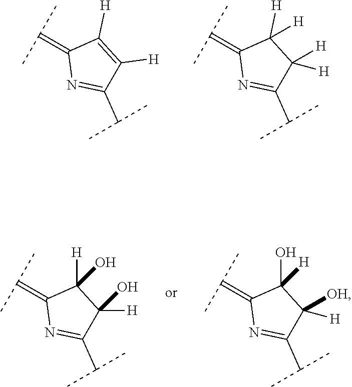
O—R¹ is a substituent in the meta or para position of the phenyl ring,
R¹ is a glyco-substituent derived from a mono-, di-, or trisaccharide group, and
each R² is independently selected from the group consisting of a linear or branched (fluoro-)alkyl group with 3 to 8 carbon atoms, phenyl, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30,
wherein
(a) in Formula 1 or 2, if B is 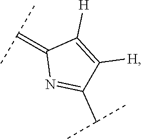
O—R¹ is a substituent in the para position of the phenyl ring and R¹ is glucosyl, then each R² is independently selected from the group consisting of a linear or branched alkyl group with 5 to 8 carbon atoms, a linear or branched fluoroalkyl group with 3 to 8 carbon atoms, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30,
(b) in Formula 1 or 2, if B is 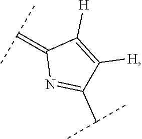
O—R¹ is a substituent in the para position of the phenyl ring and R¹ is galactosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30,
(c) in Formula 1 or 2, if O—R¹ is a substituent in the meta position of the phenyl ring and R¹ is glucosyl and R² is n-hexyl, phenyl or 3,5-bis(trifluoromethyl)phenyl, then B is 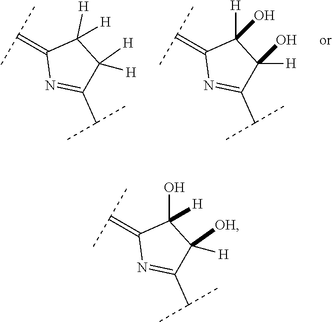
(d) in Formula 1 or 2, if O—R¹ is a substituent in the meta position of the phenyl ring and R¹ is galactosyl and R² is phenyl, then B is 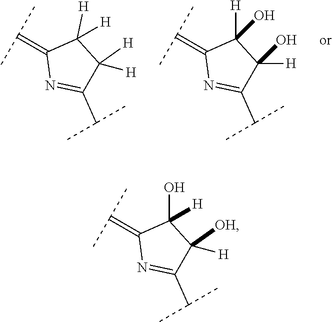
(e) in Formula 3, if B is 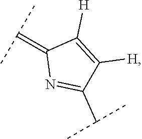
O—R¹ is a substituent in the para position of the phenyl ring and R¹ is glucosyl, then each R² is independently selected from the group consisting of a linear or branched (fluoro-)alkyl group with 5 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30,
(f) in Formula 3, if B is 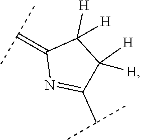
O—R¹ is a substituent in the meta position of the phenyl ring and R¹ is glucosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30, and
(g) in Formula 3, if B is 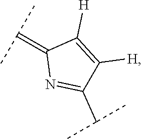
O—R¹ is a substituent in the para position of the phenyl ring and R¹ is galactosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl, meta- or para-hydroxyphenyl, meta- or para-carboxyphenyl, and meta- or para-YO-phenyl with Y being a polyethyleneglycol-residue with (CH₂CH₂O)ₙCH₃ with n=1-30.

2. The tetrapyrrolic compound according to claim 1, wherein
(a) in Formula 1 or 2, if B is 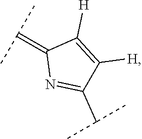
O—R¹ is a substituent in the para position of the phenyl ring and R¹ is glucosyl, then each R² is independently selected from the group consisting of a linear or branched alkyl group with 5 to 8 carbon atoms, a linear or branched fluoroalkyl group with 3 to 8 carbon atoms, 3,5-bis(trifluoromethyl)phenyl, 4-(1′-thio-β-D-glucosyl)-2,3,5,6-tetrafluorophenyl, and 4-(1′-thio-β-D-galactosyl)-2,3,5,6-tetrafluorophenyl,
(b) in Formula 1 or 2, if B is 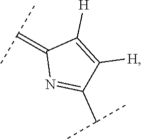
O—R¹ is a substituent in the para position of the phenyl ring and R¹ is galactosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, and 4-(1′-thio-β-D-galactosyl)-2,3,5,6-tetrafluorophenyl,
(e) in Formula 3, if B is 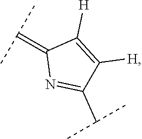
O—R¹ is a substituent in the para position of the phenyl ring and R¹ is glucosyl, then each R² is independently selected from the group consisting of a linear or branched (fluoro-)alkyl group with 5 to 8 carbon atoms, pentafluorophenyl, 3,5-bis(trifluoromethyl)phenyl, 4-(1′-thio-β-D-glucosyl)-2,3,5,6-tetrafluorophenyl, and 4-(1′-thio-β-D-galactosyl)-2,3,5,6-tetrafluorophenyl,
(f) in Formula 3, if B is 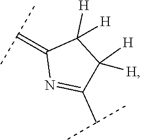
O—R¹ is a substituent in the meta position of the phenyl ring and R¹ is glucosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, and 4-(1′-thio-β-D-galactosyl)-2,3,5,6-tetrafluorophenyl, and
(g) in Formula 3, if B is 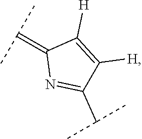
O—R¹ is a substituent in the para position of the phenyl ring and R¹ is galactosyl, then each R² is independently selected from the group consisting of a linear or branched (fluoro-)alkyl group with 3 to 8 carbon atoms, pentafluorophenyl, 3,5-bis(trifluoromethyl)phenyl, 4-(1′-thio-β-D-glucosyl)-2,3,5,6-tetrafluorophenyl, 4-(1′-thio-β-D-galactosyl)-2,3,5,6-tetrafluorophenyl.

3. The tetrapyrrolic compound according to claim 1, wherein R¹ is a glycosyl group of a mono- or disaccharide, wherein the mono- or disaccharide is preferably selected from the group consisting of glucose, galactose, mannose, ribose, fructose, rhamnose, lactose, partially deoxygenated derivatives thereof, aminosugars, such as glucosamines or galactosamines, neuraminic acids and combinations thereof.

4. The tetrapyrrolic compound according to claim 1, wherein R¹ is mannosyl or lactosyl and R² is a linear or branched (fluoro-)alkyl group with 3 to 8 carbon atoms, phenyl, pentafluorophenyl, 3,5-bis(trifluoromethyl)phenyl, 4-(1′-thio-β-D-glucosyl)-2,3,5,6-tetrafluorophenyl or 4-(1′-thio-β-D-galactosyl)-2,3,5,6-tetrafluorophenyl.

5. The tetrapyrrolic compound according to claim 4, wherein B is 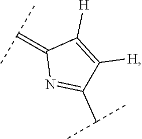
O—R¹ is a substituent in the para position of the phenyl ring and R² is phenyl.

6. The tetrapyrrolic compound according to claim 1, wherein in Formula 1 or 2 O—R¹ is a substituent in the meta position of the phenyl ring, R¹ is glucosyl and each R² is a linear or branched fluoroalkyl group with 3 to 8 carbon atoms, 3,5-bis(trifluoromethyl)phenyl, 4-(1′-thio-β-D-glucosyl)-2,3,5,6-tetrafluorophenyl or 4-(1′-thio-β-D-galactosyl)-2,3,5,6-tetrafluorophenyl.

7. The tetrapyrrolic compound according to claim 1, wherein in Formula 3 B is 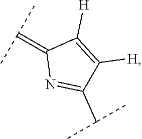
R¹ is glucosyl or galactosyl and each R² is a linear or branched (fluoro-)alkyl group with 5 to 8 carbon atoms, pentafluorophenyl, 3,5-bis(trifluoromethyl)phenyl, 4-(1′-thio-β-D-glucosyl)-2,3,5,6-tetrafluorophenyl or 4-(1′-thio-β-D-galactosyl)-2,3,5,6-tetrafluorophenyl.

8. The tetrapyrrolic compound according to claim 1, wherein in Formula 1 or 2 B is 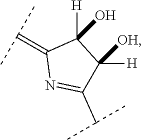
R¹ is glucosyl or galactosyl and each R² is a linear or branched (fluoro-)alkyl group with 5 to 8 carbon atoms, pentafluorophenyl, 3,5-bis(trifluoromethyl)phenyl, 4-(1′-thio-β-D-glucosyl)-2,3,5,6-tetrafluorophenyl or 4-(1′-thio-β-D-galactosyl)-2,3,5,6-tetrafluorophenyl.

9. The tetrapyrrolic compound according to claim 8, wherein R¹ is glucosyl and R² is a linear or branched alkyl group with 5 to 8 carbon atoms.

10. The tetrapyrrolic compound according to claim 1 which compound is selected from the group consisting of

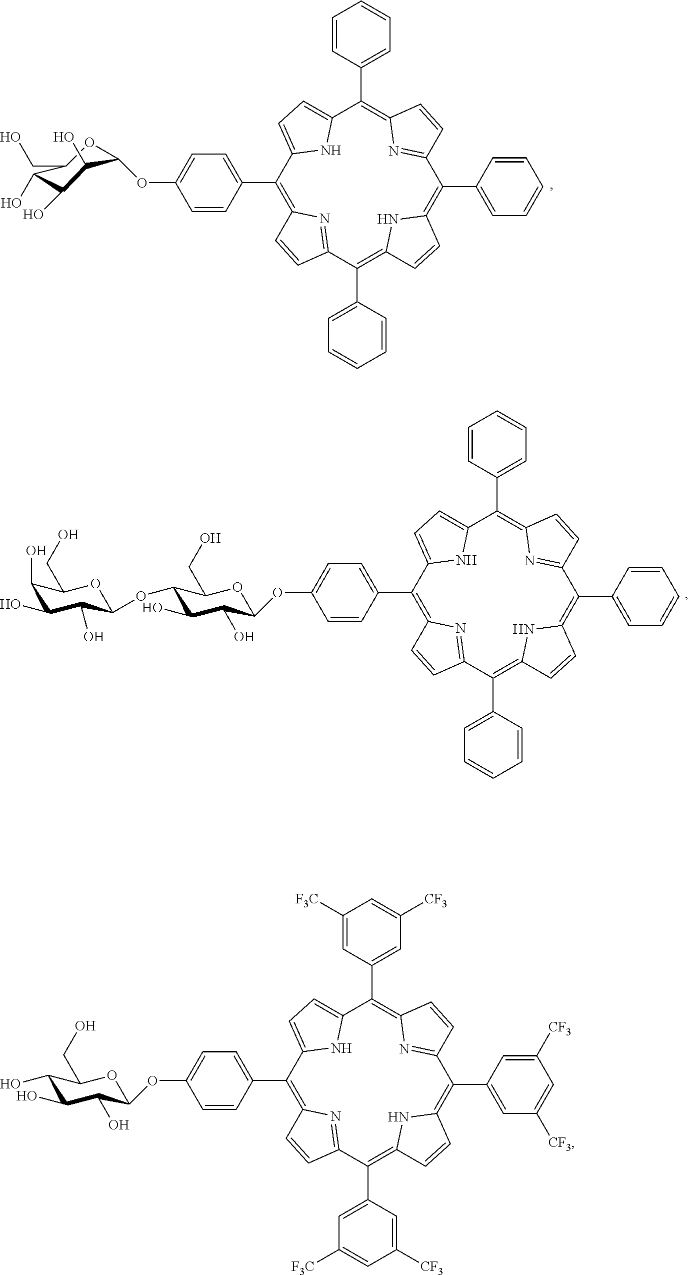

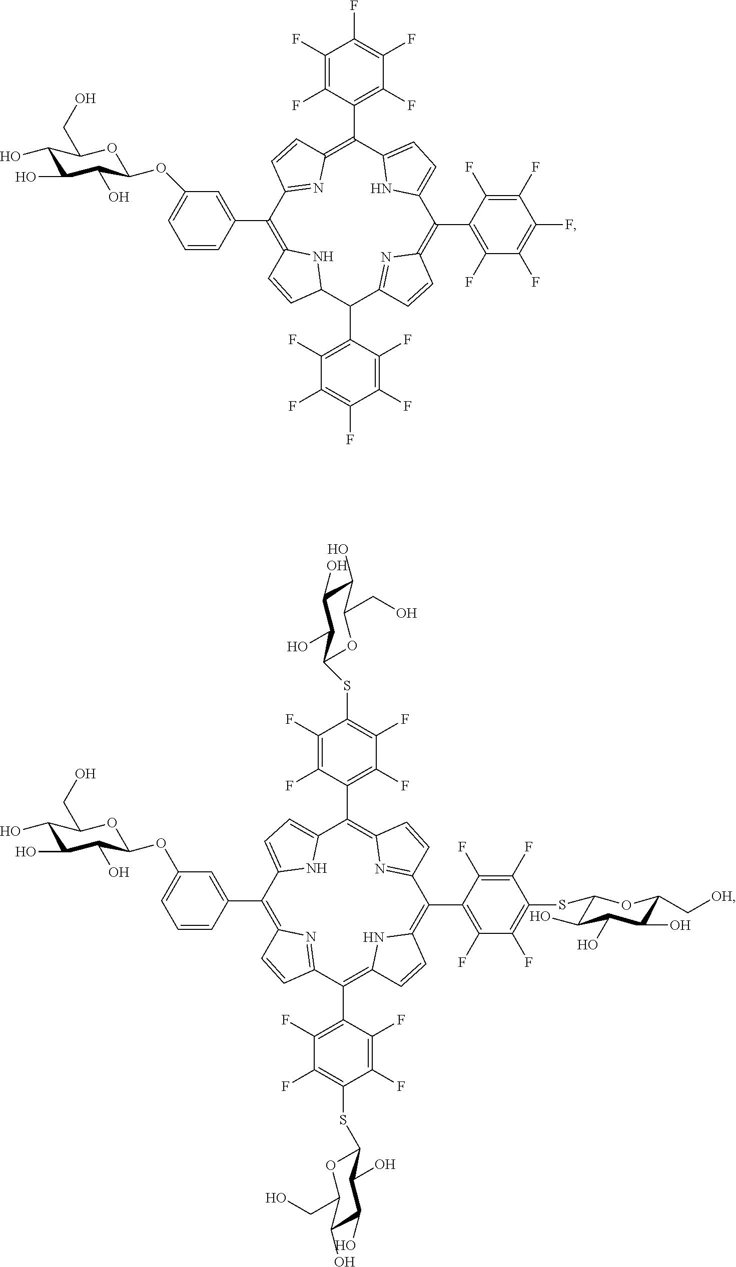

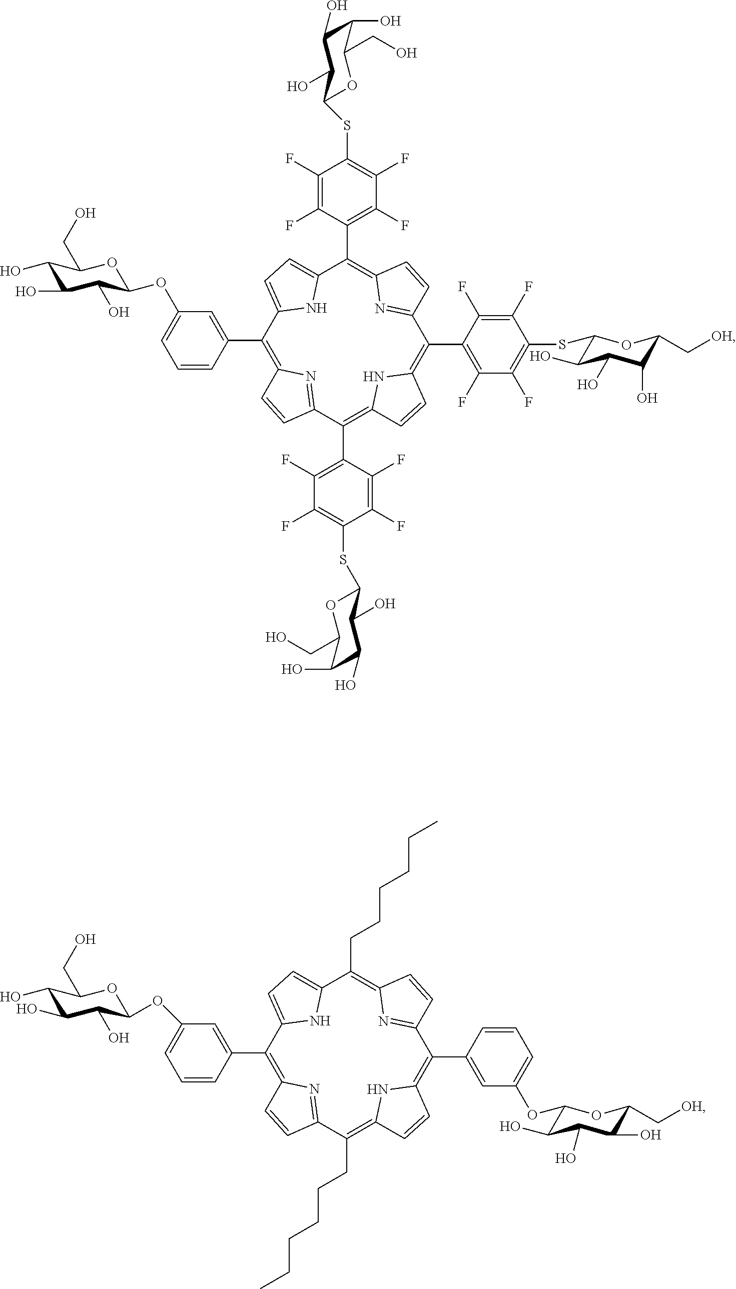

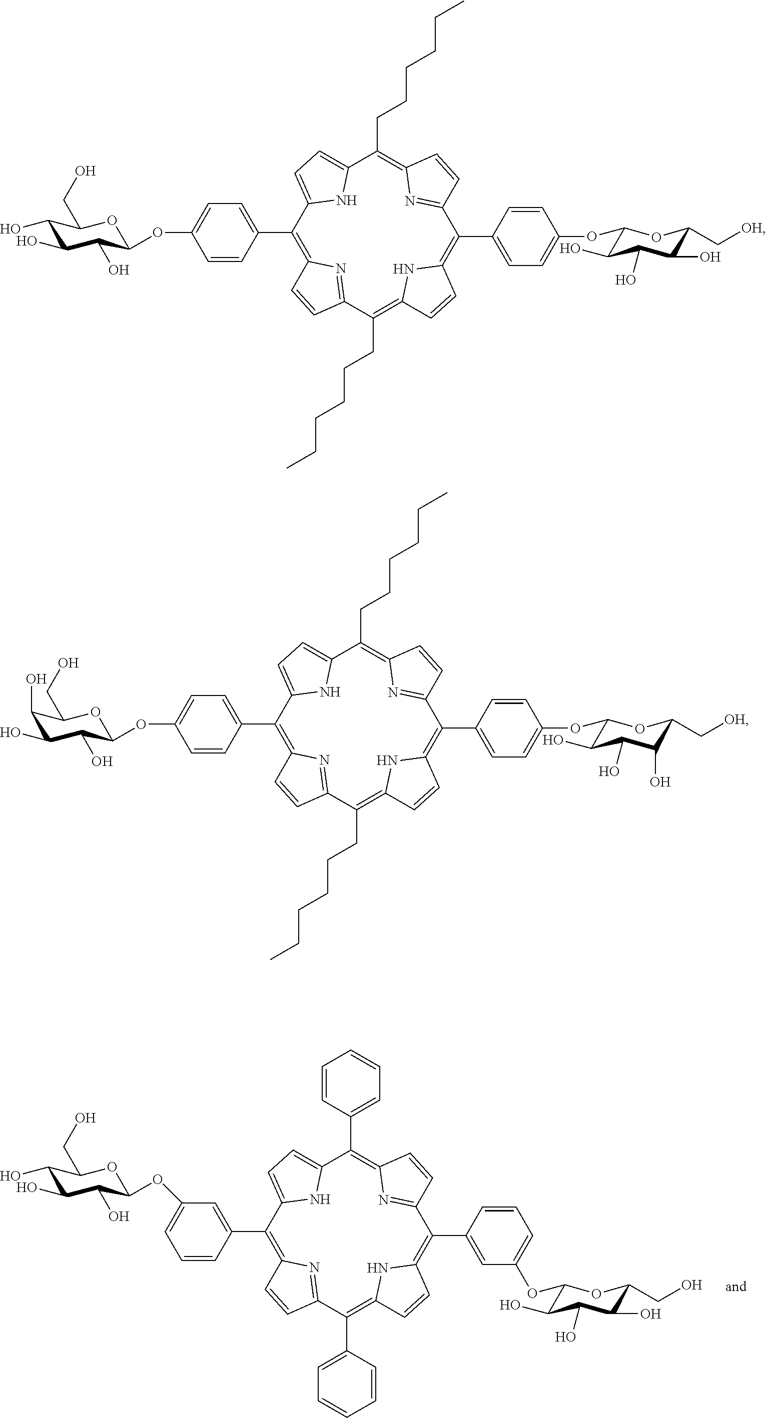

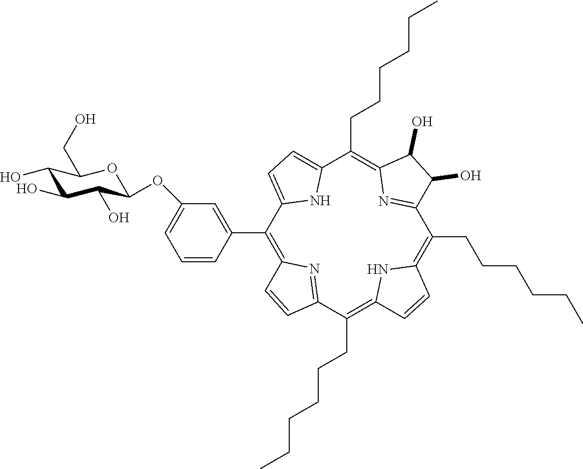

11. The tetrapyrrolic compound according to claim 10 selected from the group consisting of

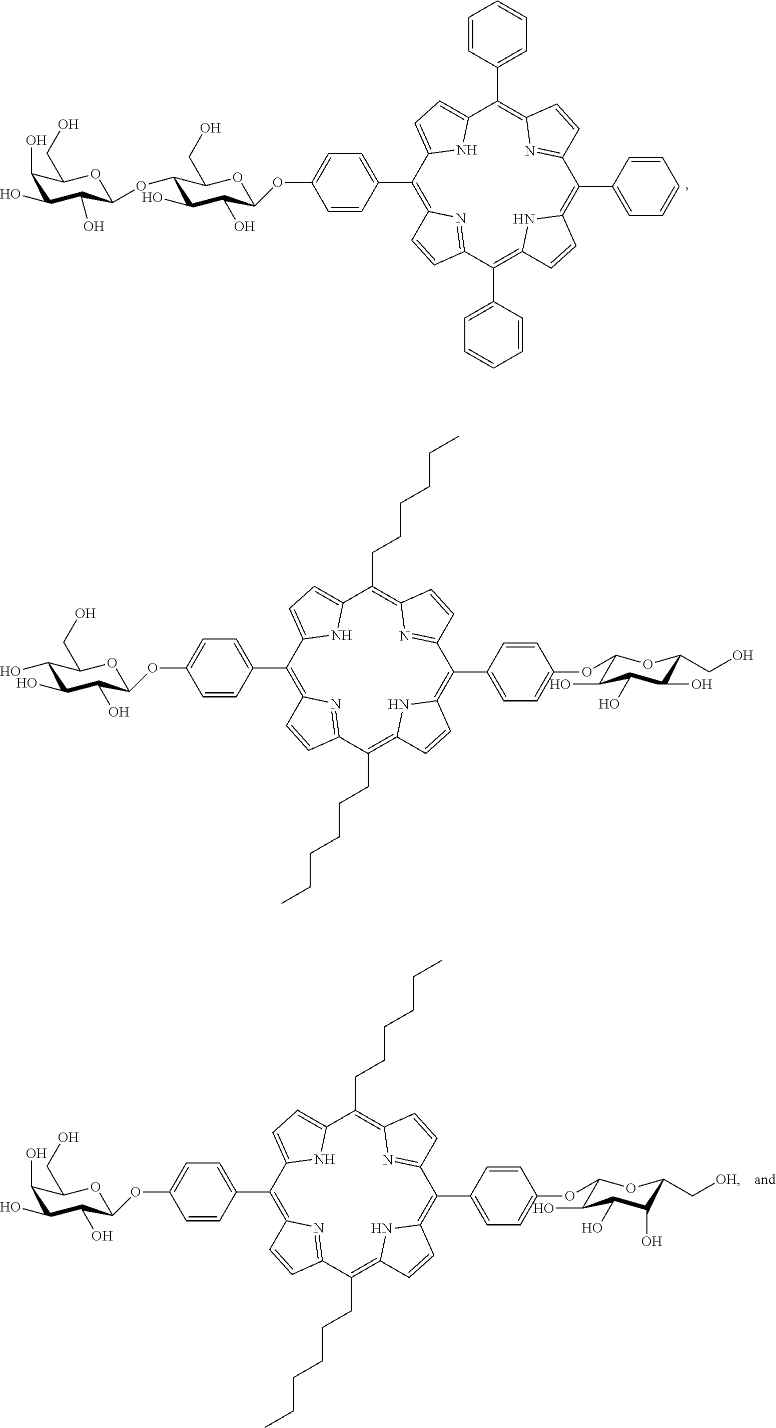

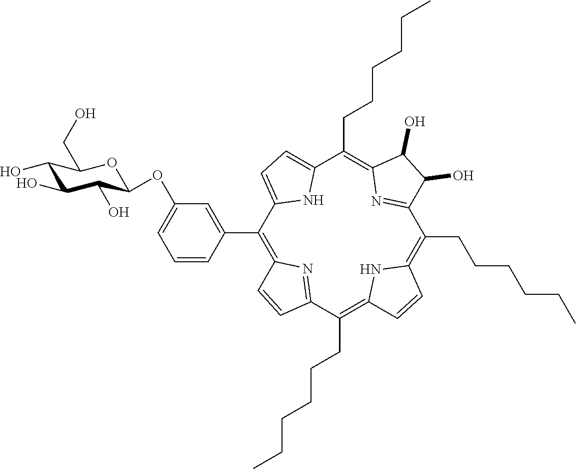

12. The tetrapyrrolic compound according to claim 1, wherein O—R¹ is a glycosidic bond.

13. A pharmaceutical composition comprising a tetrapyrrolic compound according to claim 1 wherein the pharmaceutical composition is preferably a liposomal formulation.

14. The pharmaceutical composition according to claim 13 further comprising PLGA particles, HSA particles, cyclodextrines and/or polymer particles.

15. The pharmaceutical composition according to claim 13, wherein the tetrapyrrolic compound is conjugated to a targeting agent, wherein the targeting agent is preferably selected from the group consisting of an antibody or fragment thereof.

16. A tetrapyrrolic compound according to claim 1 for use in photodynamic therapy, in particular photodynamic therapy of tumors, dermatological disorders, opthamological disorders, urological disorders, arthritis and other inflammatory diseases.

17. A tetrapyrrolic compound according to claim 1 for use in diagnosis.
